(19) United States
(12) Patent Application Publication
(10) Pub. No.: US 2024/0366702 A1
(43) Pub. Date: Nov. 7, 2024

(54) MODULATION OF SOLUBILITY, PALATABILITY, ABSORPTION, AND BIOAVAILABILITY OF MITRAGYNA SPECIOSA-DERIVED COMPOUNDS FOR ORAL AND BUCCAL DELIVERY

(71) Applicant: Syncotrance, LLC, LAS VEGAS, NV (US)

(72) Inventor: Mikhail MALAKHOV, Las Vegas, NV (US)

(21) Appl. No.: 18/607,759

(22) Filed: Mar. 18, 2024

Related U.S. Application Data
(63) Continuation of application No. 18/317,436, filed on May 15, 2023, now abandoned.
(60) Provisional application No. 63/342,098, filed on May 14, 2022.

(51) Int. Cl. A61K 36/28; A61K 9/107

(52) U.S. Cl. CPC A61K 36/28; A61K 9/1075

(57) ABSTRACT

A method of manufacture of a Kratom-derived formulation for oral, and buccal delivery comprising Mitragyna speciosa-derived compounds (e.g., mitragynine) formulated with phospholipids is disclosed. Kratom-derived extracts have dramatically improved organoleptic properties and enhanced bioavailability when formulated with phospholipids as compared to standard extracts. Also disclosed herein are methods of transmucosal administration of the formulation having phospholipids with Kratom-derived substances. The methodology is directly applicable to other botanical extracts as well as other substances requiring improved organoleptic properties.

### I. CROSS-REFERENCE TO RELATED APPLICATION

This application is a continuation application of U.S. patent application Ser. No. 18/317,436, filed May 15, 2023, which claims the benefit of U.S. Provisional Patent Application No. 63/342,098, filed May 14, 2022, which is incorporated by reference herein in its entirety.

### II. BACKGROUND

Mitragyna speciosa, or Kratom, is an ethnomedicinal tree native to Malaysia. It has traditionally been consumed as a leaves decoction for its stimulant effects to counter fatigue. Additionally, it has been employed, as a leaves poultice, to treat fever, diarrhea, and wound healing. The other reported pharmacological properties of M. speciosa, include anesthetic, antinociceptive, analgesic, and stimulant effects.

The pharmacological effects of M. speciosa are mainly attributed to its principal alkaloid mitragynine. Kratom is consumed in many different forms such as tea, powder, capsules, tablets, tinctures, nanoemulsions, extracts, etc. Kratom taste is very unpleasant and aversive and most frequently described as bitter, acrid, earthy, grassy, and/or pungent.

Reducing aversive flavors, irritation, and especially bitterness can significantly improve the Kratom regimen experience. Delivering Kratom in lipophilic and neutral/basic formulations will improve stability and bioavailability.

Mitragynine is an indole alkaloid comprising 66% of total alkaloids in extracts of Mitragyna speciosa leaves (Adkins, J. E., Boyer, E. W., McCurdy, C. R., 2011. Mitragyna speciosa, a psychoactive tree from Southeast Asia with opioid activity. Curr. Top. Med. Chem. 11, 1165-1175) (Ponglux, D., Wongscripipatana, S., Takayama, H., Kikuchi, M., Kurihara, M., Kitajima, M., Aimi, N., Sakai, S., 1994. A new indole alkaloid, 7 alpha-Hydroxy-7H-mitragynine, from Mitragyna speciosa in Thailand. Planta Med. 60, 580-581.). Mitragynine is a weak base (pKa 8.1), lipophilic (Log P=1.73), with low aqueous solubility (64.6±1.2 μg/ml) (Ramanathan S, Parthasarathy S, Murugaiyah V, Magosso E, Tan S C, Mansor S M. Understanding the physicochemical properties of mitragynine, a principal alkaloid of Mitragyna speciosa, for preclinical evaluation. Molecules 2015; 20:4915-4927). Its solubility in an aqueous media is pH dependent; at pH 4 and 7, mitragynine would be dissolved with concentration levels of 130 μM (51.8 μg/ml) and 83 μM (33.1 μg/ml), respectively (Kong, W. M., Chik, Z., Mohamed, Z., Alshawsh, M. A., 2017. Physicochemical characterization of Mitragyna speciosa alkaloid extract and Mitragynine using in vitro high throughput assays. Comb. Chem. High Throughput Screen. 20, 796-803.). This is significantly below the concentration of a typical Kratom shot (8-11 mg/ml); therefore, various solubility enhancers or co-solvents (e.g., propylene glycol and glycerol) are commonly used. The compound is moderately stable at neutral pH (˜3.5% degradation after 3 h) but degraded at pH 1.2 (by 26% degradation after 1-2 hr) (Manda, V. K., Avula, B., Ali, Z., Khan, I. A., Walker, L. A., Khan, S. I., 2014. Evaluation of in vitro absorption, distribution, metabolism, and excretion (ADME) properties of mitragynine, 7-hydroxymitragynine, and mitraphylline. Planta Med. 80, 568-576) (Ramanathan, S., Parthasarathy, S., Murugaiyah, V., Magosso, E., Tan, S. C., Mansor, S. M., 2015. Understanding the physicochemical properties of mitragynine, a principal alkaloid of Mitragyna speciosa, for preclinical evaluation. Molecules 20, 4915-4927). Mitragynine can cross the blood-brain barrier and has adequate exposure to the brain (% AUC_{brain}/AUCₚₗₐₛₘₐ, 65.8±4.5%) in rats (Kong W M, Mohamed Z, Alshawsh M A, Chik Z. Evaluation of pharmacokinetics and blood-brain barrier permeability of mitragynine using in vivo microdialysis technique. J Pharm Biomed Anal 2017; 143:43-47).

The absolute oral bioavailability of mitragynine studied in rats in comparison to intravenous administration, was 17.0, 25.1, and 31.2% for pure mitragynine hydrochloride, lyophilized Kratom tea (a traditional preparation), and the lyophilized Kratom tea organic fraction, respectively (Avery B A, Boddu S P, Sharma A, Furr E B, Leon F, Cutler S J, McCurdy C R. Comparative Pharmacokinetics of Mitragynine after Oral Administration of Mitragyna speciosa (Kratom) Leaf Extracts in Rats. Planta Med. 2019 March; 85 (4): 340-346). Lyophilized Kratom tea organic fraction appears similar in its preparation and composition to dry powder Kratom extracts offered by commercial sources and used for research and development purposes. Multiple factors might be responsible for the increased mitragynine exposure when the animals were dosed with the Lyophilized Kratom tea and the Lyophilized Kratom tea organic fraction compared to the mitragynine hydrochloride dose alone, including the possible presence of permeability/solubility enhancers, cytochrome P450 (CYP450) enzyme inhibitors, and/or gastrointestinal motility inhibitors in Lyophilized Kratom tea and the Lyophilized Kratom tea organic fraction (Jaiswal S, Sharma A, Shukla M, Vaghasiya K, Rangaraj N, Lal J. Novel preclinical methodologies for pharmacokinetic drug-drug interaction studies: spotlight on “humanized” animal models. Drug Metab Rev 2014; 46:475-493). It must be noted that to this day, while mitragynine is considered to be the major antinociceptive compound, some data suggest that pharmacokinetics of 7-Hydroxymitragynine is more relevant (Kruegel A C, Uprety R. Grinnell S G, Langreck C, Pekarskaya E A, Le Rouzic V, Ansonoff M, Gassaway M M, Pintar J E, Pasternak G W, Javitch J A, Majumdar S, Sames D. 7-Hydroxymitragynine Is an Active Metabolite of Mitragynine and a Key Mediator of Its Analgesic Effects. ACS Cent Sci. 2019 Jun. 26; 5 (6): 992-1001). Although a more recent study suggests that mitragynine is still the biggest contributor to antinociception (Berthold E C, Kamble S H, Raju K S, Kuntz M A, Senetra A S, Mottinelli M, Leon F. Restrepo L F, Patel A, Ho N P, Hiranita T, Sharma A, McMahon L R, McCurdy C R. The Lack of Contribution of 7-Hydroxymitragynine to the Antinociceptive Effects of Mitragynine in Mice: A Pharmacokinetic and Pharmacodynamic Study. Drug Metab Dispos. 2022 Feb; 50 (2): 158-167).

Similar bioavailability of ˜21% resulting from an oral administration in rats was calculated from other studies by Ya et al. (Ya K, Tangamornsuksan W, Scholfield C N, Methaneethorn J, Lohitnavy M. Pharmacokinetics of mitragynine, a major analgesic alkaloid in Kratom (Mitragyna speciosa): A systematic review. Asian J Psychiatr. 2019 June; 43:73-82).

Comparative analysis of pharmacokinetics of orally administered lyophilized Kratom tea (a traditional preparation) versus commercial liquid Kratom shot (OPMS liquid Kratom, Optimized Plant Mediated Solutions, Choice Organics, Los Angeles, CA) revealed that commercial Kratom extract (OPMS liquid shot) has resulted in the delayed and greater systemic exposure of Kratom alkaloids compared to lyophilized Kratom tea possibly due to the differences in the composition of other constituents (Kamble S H, Berthold E C, King T I, Raju Kanumuri S R, Popa R, Herting J R, Leon F, Sharma A, McMahon L R, Avery B A, McCurdy C R. Pharmacokinetics of Eleven Kratom Alkaloids Following an Oral Dose of Either Traditional or Commercial Kratom Products in Rats. J Nat Prod. 2021 Apr. 23; 84 (4): 1104-1112). OPMS Kratom shot, judging by its highly aversive organoleptic properties and rich pigmentation is likely prepared from the dry powder extract with approximately 45-70% mitragynine content, leaving many coextracting and copurifying substances. Flavonoids, especially flavonol groups, present in Kratom extracts, have also been reported to be responsible for the antinociceptive effect. For example, quercetin is reported to bind to the a 2-adrenergic receptors of the central and peripheral nervous systems and has a dose-dependent antinociceptive effect. In addition, quercetin and its derivative, rutin, also activate the cGMP/PKG/ATP-sensitive potassium pathway and deactivate cellular regulating proteins in neurons to block pain and inflammation reactions with fewer side effects. Taken together, the agonist, antagonist, synergistic interactions among alkaloids, flavonols, and other active constituents acting on multiple targets could have collectively exerted the antinociceptive effect. (Goh Y S, Karunakaran T, Murugaiyah V. Santhanam R. Abu Bakar M H, Ramanathan S. Accelerated Solvent Extractions (ASE) of Mitragyna speciosa Korth. (Kratom) Leaves: Evaluation of Its Cytotoxicity and Antinociceptive Activity. Molecules. 2021 Jun. 17; 26 (12): 3704).

Due to its lipophilicity and poor water solubility at physiological pH, mitragynine can be classified as a Class II Drug according to the Biopharmaceutical Classification System (BCS). Dissolution is one of the major factors influencing mitragynine oral bioavailability. A slow dissolution rate may play an important role in its low bioavailability (Barthe, L., Woodley, J., Houin, G., 1999. Gastrointestinal absorption of drugs: methods and studies. Fundam. Clin. Pharmacol. 13, 154-168). Currently available liquid commercial preparations of Kratom may help resolve this issue by delivering a solubilized Kratom. However, most formulations are likely to precipitate upon mixing with saliva before they even reach gastrointestinal fluids.

The limited oral bioavailability of mitragynine, especially in its purified form, and greater systemic exposure achieved with Kratom extracts containing coextracting/copurifying substances indicate the need to develop formulations that can both improve the bioavailability of mitragynine and Kratom extracts and organoleptic properties of said extracts.

### III. SUMMARY

The pharmacological effects of Mitragyna speciosa are mainly attributed to its principal alkaloid mitragynine.

Mitragynine is a lipophilic alkaloid, as indicated by a logP value of 1.73. Mitragynine had poor solubility in water and basic media. Mitragynine is soluble in acidic environments, but it is acid labile. The hydrophobicity, poor water solubility, high variability of drug release in simulated biological fluids and acid degradable characteristics of mitragynine probably explain the large variability of its pharmacological responses reported in the literature.

Kratom is consumed in many different forms such as tea, powder, capsules, tablets, tinctures, nanoemulsions, extracts, etc. Kratom taste is very unpleasant and aversive and generally described as earthy, bitter, acrid, grassy, and/or pungent. Reducing aversive flavors, and especially bitterness can significantly improve Kratom regimen experience.

Some commercially available formulations are solutions, suspensions, emulsions, or nanoemulsions, sold as 10-30 ml “shots.” Compared to standard tinctures, extracts, and other forms such as powders, teas, and capsules, nanoemulsions are claimed to have faster onset, improved potency, and bioavailability. One example of such product is Hush Nano Kratom Shot manufactured by Hush Worldwide LLC, 30 N Gould St Ste R, Sheridan, WY, 82801, USA.

Similarly to many other botanical supplements, Kratom leaf powders, teas, and infusions taste bitter, herbal, and astringent. Concentrated Kratom extracts in the form of powders and concentrated liquid forms are particularly unpalatable and have many aversive sensory attributes. Acute bitterness, similar to that produced by caffeine, quinine, antibiotics, and other natural and synthetic pharmaceuticals, is one of the most frequently cited organoleptic characteristics of Kratom. The present evaluation of organoleptic properties of Kratom revealed that bitterness is not the only significant concern. Some of the other flavor-aversive sensory attributes frequently cited by Kratom users are as follows: sour/acidic, burnt, burnt leaves, dried leaves, chlorophyll, grassy, green, peppery, hay, orange peel, metallic, dry/pucky/astringent, and earthy. Sour/acidic taste is also strongly irritating and sometimes referred to by users as the Kratom taste. Both dry powders and liquid Kratom extracts carry off-flavors that produce trigeminal irritation on the tongue, soft palate, epiglottis, larynx, and pharynx. The irritation is described as tingling, burning, and numbing. Numbing is related to the analgesic/antinociceptive effect of Kratom (Swogger M T, Walsh Z. Kratom use and mental health: A systematic review. Drug Alcohol Depend. 2018 Feb. 1; 183:134-140). Regular users of Kratom report a spasm-like feeling at the back of the tongue and soft palate that may manifest even when they only think about Kratom or are about to take a Kratom drink, suggesting a conditioned reflex.

For brevity, the aversive sensory attributes from hereon will be referred to as off-flavors and will also include off-notes (malodor) and off-tastes, although it should be understood that off-flavors (e.g., fishy) and off-tastes (e.g., bitter) are different in nature and act on different types of receptors.

The identities and intensities of off-flavors in various Kratom preparations are product specific, meaning that some Kratom extracts are less aversive than others, and concentration dependent, meaning that with a higher dilution in the drink or edible preparation, the off-flavors are easier to mask.

Currently, most Kratom is still consumed as a dry powder extract in capsule form. The disadvantage of capsules is slow onset—it takes about one hour after capsule ingestion to feel pain relief. Some liquid preparations are reported to relieve pain in as quickly as 15 minutes. To minimize exposure to the aversive taste of liquid Kratom the industry adopted an approach of creating highly concentrated extracts where 125-250 mg of mitragynine is delivered in a 10-15 ml volume, commonly referred to as a “shot,” so that a consumer can ingest it as quickly as possible and chase it with a sweetened flavored drink to rinse the mouth. This still induces a strong instant aversive reaction, and the aftertaste of most commercial preparations is strong, unpleasant, and lingers for several minutes.

The strong aversive taste of liquid Kratom is commonly discussed among Kratom users and Kratom manufacturers. Many of the manufacturers invested heavily in attempts to produce palatable formulations of Kratom. These attempts, however, seem to be limited to standard bitter maskers/blockers, overwhelming levels of high intensity sweeteners, and strong overbearing flavors. This type of approach may be successful with many other bitter botanicals, supplements, vitamins, energy drinks, and the like. However, most Kratom users admit that there are no palatable Kratom shots on the market. The present organoleptic analysis of over 40 commercial liquid Kratom preparations confirmed that the industry, thus far, has not been successful in producing a palatable Kratom shot. The most palatable products identified were Tusk brand products produced by Monster Vape Labs, 1041 Crews Commerce Dr., Unit 100, Orlando, FL 32837, www.tuskKratom.com. The product's List of Ingredients suggests that the manufacturer uses a purified form of mitragynine and the formulation itself is 5.26 mg/ml which is about half the concentration of a typical Kratom shot 8-11 mg/ml.

One other aspect of Kratom's aversive taste and lingering, highly unpleasant aftertaste is precipitation of the extract on the tongue. Mitragynine itself has a very limited solubility (64.6±1.2 μg/mL) in a saliva type fluid pH 6.5 (Ramanathan S, Parthasarathy S, Murugaiyah V. Magosso E, Tan S C, Mansor S M. Understanding the physicochemical properties of mitragynine, a principal alkaloid of Mitragyna speciosa, for preclinical evaluation. Molecules 2015; 20:4915-4927). Therefore, it should be expected that upon oral administration of a 10 mg/ml Kratom shot solution (more that 150× excess over solubility), and the resulting dilution of the co-solvents in saliva, mitragynine and other tastants in the extract will abruptly precipitate. To prevent precipitation, the tastants are associated into stable complexes that will also physically prevent interaction with receptors and will not dissociate. Therefore, it is highly desirable to develop a system where most, or at least some, of the product is physically removed via formation of complexes (with lipids generally, and phospholipids more specifically). These complexes themselves must be stable enough to survive dilution with saliva long enough to be fully ingested and rinsed away. Encapsulation or complexation is rarely 100% efficient and removal of unincorporated tastants requires additional processing step(s), such as diafiltration or chromatography, which can be cost-prohibitive. Therefore, to neutralize the residual, unincorporated tastants in the product, sweeteners, taste-maskers, and flavors are utilized.

Dry powder Kratom extracts offered by various manufacturers vary in mitragynine content and total alkaloid content from as low as 38% up to 90%. Currently there are no established or harmonized standards for raw materials, extraction/purification methodology, purity or impurities profile, or final products. The only parameters that are being monitored are those common to all food and supplements and enforced by the FDA, namely heavy metals, microbial burden, and residual solvents. A typical COA will also list 3-7 related alkaloids, e.g., 7-hydroxymitragynine, Paynantheine, Speciogynine, Speciociliatine, Mitraphylline, Isorhynchophylline, and Corynoxine. Extracts that have higher presence of related alkaloids and other phytochemicals, are referred to as “full spectrum” and manufacturers imply that such extracts are more psychoactive and analgesic/antinociceptive. This seems to be supported by research for copurifying flavonols, but it is not clear how other related alkaloids add to psychoactive properties). Extracts of the same/similar mitragynine and total related alkaloid content, whether low or high purity, may differ significantly from each other in terms of overall chemical composition (coextracting/copurifying substances), color, appearance, and solubility profile. Organoleptic properties of such extracts with same/similar mitragynine content may differ dramatically. It is also not uncommon that different batches of the same product from the same manufacturer (standardized by the manufacturer to the same mitragynine %) come in different colors, organoleptic properties, and solubility. Currently, there is no requirement or convention for extract manufacturers to characterize their products for coextracting/copurifying substances. Consequently, coextracting/copurifying substances affecting organoleptic properties of Kratom extracts have not been characterized. Specification sheets and COAs lack this information and liquid shot manufacturers willing to mask off-flavors must act in a purely empirical manner.

Extracts with a higher mitragynine percentage typically have fewer off-flavors and are easier to formulate into a palatable form. As expected, lower percentage extracts are typically more bitter and irritating and have more chlorophyll, burnt, earthy, orange peel notes, which are particularly difficult to mask. Most remarkably, pure mitragynine, despite being an alkaloid, which is known to be bitter in general, is relatively tasteless. Organoleptic properties of extracts of 45, 55, 70 and 90% mitragynine (see Example A) were analyzed and found that solutions containing the same concentration of mitragynine differ dramatically from each other, confirming that mitragynine itself may not be the main contributor of bitterness, irritation, and other off notes, but the substances that coextract/copurify with mitragynine.

Example A demonstrated that the purer forms are preferred. However, each additional purification step adds to the cost of extract and translates to the higher cost of final product. Also, as follows from the research cited above, the less pure extracts are more bioavailable and result in higher systemic exposure and thus are more efficacious.

There is a need, therefore, to develop a platform technology easily customizable to various Kratom extracts, ideally a universal formulation, to produce Kratom shots, drinks, and edibles with acceptable palatability, which will allow the use of less pure, thus less expensive, and more potent, extracts. The formulation itself and manufacturing technology (including masking agents, excipients, other materials, equipment, and manufacturing process) must add as little as possible to the cost of final product.

There is also a need for universal formulation (platform technology) to be used with any botanical extract.

There is also a need for universal formulation (platform technology) to be used with any unpalatable mineral, vitamin, dietary supplement, or food.

There is also a need for universal formulation (platform technology) to be used with any unpalatable pharmaceutical.

As will be further demonstrated in the description, the following factors were identified as contributors to palatability of Kratom formulations:
1) Overall purity of the extract (% mitragynine), Example A. Typically, the higher the percentage of mitragynine in the extract, the fewer other tastants (off-flavors and irritants) will be present in the final products.
2) Type of extract. Different extracts contain different types and amounts of impurities or tastants, some of which may be particularly aversive and/or difficult to mask. It can be reasonably inferred that these tastants are also alkaloids that coextract and copurify with mitragynine due to their similar chemical structure and/or physico-chemical properties. Alkaloids in general are known to be very bitter and astringent.
3) Concentration of the extract in final product. Diluted products will have reduced interaction of tastants with receptors and impart fewer off-flavors.
4) pH of the preparation. Mitragynine is significantly more soluble at pH<5.0, therefore all commercially available Kratom preparations or “shots” are in the pH range of 3.0-5.0. Solubility, of course, is dependent on the concentration and at lower concentrations of mitragynine it may be possible to formulate at pH above 5.0. However, these diluted drinks are perceived to be less potent, less convenient, and less desirable. Several formulations were titrated to pH 4.1-10.1, Example K. pH range 6.0-8.0 delivered the most neutral organoleptic profile and was largely devoid of any irritation and tingling associated with formulations at pH<6.0.
5) Type of acidulant. Unless the formulation is composed predominantly of an organic solvent, an acidulant must be used to acidify the solution when converting dry powder extract into liquid form. In most cases, acidic pH must also be maintained in the final ready-to-drink product. Most commercial formulations use citric and/or ascorbic acid, and sometimes malic acid, as acidulants. These acids are known to create pleasant tartness and thus commonly used in the formulation of food products where tartness is desirable. It was determined that these acids, possibly via the tartness imparted, enhance sour/acidic off notes of Kratom. These acids also enhance the irritation felt in the oral cavity in general and particularly under the tongue and on the sides of the tongue, especially in frequent users. Contrary to that, phosphoric acid, also commonly used in food products, did not enhance sour/acidic off notes, helped reduce the irritation, and delivered better flavor and mouthfeel, even in flavors typically associated with tartness, e.g., passion fruit, Example G.
6) Presence of organic solvents in the formulation. Mitragynine is poorly soluble in aqueous solutions, therefore many marketed products are formulated with organic solvents such as Propylene Glycol, Polyethylene glycols (e.g., PEG-300, PEG-400), glycerol, other polyols, ethanol, sugar alcohols, etc. Whereas glycerol and polyols generally impart sweet taste to a formulation, Propylene Glycol and PEGs, may cause irritation and enhance off-flavors. Glycerol at concentrations above 10% may cause undesirable warming sensation in the mouth and enhance off-flavors. Alcohol may cause burning sensation and enhance off-flavors. The effects of these compounds on taste will strongly depend on the concentration used and the general matrix.
7) Sweetness level. It is generally known that sweet taste can efficiently suppress bitterness. However, most commercial Kratom preparations are too bitter to rely on sweetness alone as a taste masker. Even saturated solutions of sucrose (52% w/v) are not able to mask bitterness, other off-flavors, and irritation caused by most Kratom extracts. Sweetness may also be enhanced by specialized sweetness enhancers and taste modulators (e.g., adenosine, hesperetine).
8) Type of sweetener. Sucrose (table sugar) is the common sweetener in all taste masking applications due to its natural sweetness and low cost. However, it may not be enough to use sucrose alone to suppress bitterness of Kratom extracts. Sucrose is also calorigenic and cariogenic, causes strong glycemic response and must be avoided by diabetics. High intensity sweeteners (HIS), sometimes combined with low intensity non-caloric sweeteners (e.g., erythritol, allulose, sorbitol, xylitol, etc.) are commonly used to formulate sugar free or reduced sugar foods, supplements, and pharmaceuticals. Some of the HIS however, may enhance irritation and off-flavors caused by Kratom extracts and should not be used. It has been found that sucralose and stevia, present in some commercial Kratom products, enhance irritation and must be avoided.
9) The biggest contributor to improved palatability of Kratom, as abundantly demonstrated in present teaching was the presence of phospholipids. Examples K. L.

Other factors directly related to palatability and more specific to the flavor are as follows:
10) General taste maskers. Fats/oils, phospholipids, carbohydrates, and other compounds are known to reduce the interaction of tastants with receptors in oral cavity.
11) Specialized taste maskers, developed by flavor manufacturers for masking very specific off-flavors. The maskers identified in screens presented in Examples B and D are examples of such maskers. OSF 7186B was designed for masking bitterness, green/grassy, and other off flavors, whereas OSF 6754B PDR was useful at reducing epiglottis irritation among other off flavors.
12) Flavors. To achieve better palatability, liquid Kratom products are typically flavored with natural or artificial flavors. The presence of a flavor, e.g., coffee or chocolate, can help the brain focus on the flavor and associate the off-flavors, e.g., bitterness and roasted notes with the coffee. Examples B, D.
13) Mouthfeel enhancers, taste modifiers, taste modulators, flavor enhancers, etc. As will be discussed in detail below, neohesperidin dihydrochalcone and similar flavanones are examples of such taste modulator with sweetening and masking properties particularly applicable to masking Kratom extracts.

Several of the marketed liquid Kratom shots include the word “Nano” on their packaging, implying use of some form of nanotechnology, delivery of Kratom extract and mitragynine as nanoemulsions, incorporation of mitragynine in the nanospheres, improved bioavailability, faster onset, stronger pharmacological action, improved therapeutic effect, and the like. Specific examples of such brands are Hush, Amazing Botanicals, MIT Therapy, MIT Wellness, Mitra 165 and others. Lists of ingredients on the bottles/packages of these brands/products do not support such claims (at the minimum one would expect the use of lipids and/or surfactants or polymers in the formulation) and analysis of these and other products by dynamic light scattering (DLS) failed to detect any particles in the nano range.

Still other benefits and advantages of the present subject matter will become apparent to those skilled in the art to which it pertains upon a reading and understanding of the following detailed specification.

### IV. DETAILED DESCRIPTION

Formulations presented in the present teaching, due to presence of phospholipids, and other lipids where liquid lecithins are used, were expected to form nanoemulsions. Specifically, the nanoparticles are expected to be formed by phospholipid monolayer with polar heads exposed to aqueous solution and fatty acid tails hidden inside of the nanosphere. Dynamic light scattering (DLS) experiments demonstrated presence of nanoparticles 100-500 nm in diameter and filtration of the formulations though 0.22 μm filter demonstrated that particles can be further reduced in size (see Example K). Other techniques known to reduce particle size, rearrange structure of the particles, and improve entrapment or encapsulation efficiency include sonic oscillation, high pressure or high sheer mixing, cavitation, etc. The experiments also demonstrated that up to 88% of mitragynine present in the formulations of the present teaching were included in nanoparticles.

Another aspect of the present teaching is phospholipids' and/or nanoparticles' ability to maintain solubility of mitragynine (and other compounds/tastants of Kratom extract) at pH above 5.0. Without adhering to any specific molecular mechanism that may narrow the scope of the present teaching, based on experimental data, the improved solubility is achieved via (partial) removal of mitragynine, and other compounds/tastants of the extract, from the aqueous phase and incorporation of these compounds into the nanoparticles. Example K provides a demonstration of this point.

The physical removal of tastants from the aqueous phase is not the only contributor to the improved organoleptic properties of the formulations of the present teaching. Lipids in general, and phospholipids in particular, may bind to receptors in the oral cavity and reduce the exposure of the receptors to tastants (Nakamura T, Tanigake A, Miyanaga Y, Ogawa T, Akiyoshi T, Matsuyama K. Uchida T. The effect of various substances on the suppression of the bitterness of quinine-human gustatory sensation, binding, and taste sensor studies. Chem Pharm Bull (Tokyo). 2002 Dec; 50 (12): 1589-93).

Example K demonstrates that liquid lecithin at 6% (containing 50% phospholipids and 50% sunflower oil, i.e., final concentration of phospholipids 3%) was a more efficient Kratom taste masker than de-oiled lecithin at 4% (97% phospholipids), final concentration of phospholipids 4%. Dry powder premix containing only de-oiled lecithin was a less efficient masker than dry powder premix containing de-oiled lecithin and powdered sunflower oil Example M. Therefore oils/fats can further contribute to the taste masking properties of phospholipids.

Another aspect of this improved solubility at elevated pH is improved organoleptic properties of the formulation while maintaining physical stability of the formulation (absence of phase separation).

Phospholipids' contribution to taste masking, therefore, appears to work via at least a triple mechanism of action: physical encapsulation of tastants, coating/blocking of the receptors, improved solubility of tastants permitting formulation at neutral and basic pH. Additional mechanisms contributing to improved palatability by phospholipids are possible.

Another aspect is that formulations of the present teaching were obtained using standard equipment common to general research and development laboratories. Lab scale batches up to two liters were produced using regular magnetic stirrer, such as VWR Scientific Model 660, and process development and scale-up batches up to 200 liters were produced using Caframo 1540 Variable-speed Stirrer. The technology does not require use of high pressure mixer, sonic oscillator (sonicator), pressure cell, cavitation cell, or the like. Use of such equipment may shorten production time, improve throughput, reduce particle size, etc., but is not required to obtain physically stable nanoemulsions with improved organoleptic properties as claimed in the present teaching.

Phospholipids in the form of Drug-Phospholipid complexes, emulsions, liposomes, micelles, etc., have been used in drug delivery for a number of specific purposes such as improved permeation of blood brain barrier (Li B, Han L, Cao B, Yang X, Zhu X, Yang B, Zhao H, Qiao W. Use of magnoflorine-phospholipid complex to permeate blood-brain barrier and treat depression in the CUMS animal model. Drug Deliv. 2019 Dec; 26 (1): 566-574), improved oral bioavailability (Tan Q, Liu S, Chen X, Wu M. Wang H, Yin H, He D. Xiong H, Zhang J. Design and evaluation of a novel evodiamine-phospholipid complex for improved oral bioavailability. AAPS PharmSciTech. 2012 June; 13 (2): 534-47), reduction of gastrointestinal toxicity (Lanza F L, Marathi U K, Anand B S, Lichtenberger L M. Clinical trial: comparison of ibuprofen-phosphatidyl choline and ibuprofen on the gastrointestinal safety and analgesic efficacy in osteoarthritic patients. Aliment Pharmacol Ther. 2008 Aug. 15; 28 (4): 431-42).

Phospholipids have a high degree of biocompatibility and are deemed ideal pharmaceutical excipients in the development of lipid-based drug delivery systems. Their unique features include permeation, solubility enhancement, emulsification, micellization, etc. Phospholipids can be used in a variety of pharmaceutical drug delivery systems. The primary usage of phospholipids in a colloidal pharmaceutical formulation appears to be their ability to enhance the bioavailability of a drug with low aqueous solubility (BCS Class II drugs), and to enhance membrane penetration of BCS Class III drugs, drug uptake and release enhancement or modification, protection of sensitive active pharmaceutical ingredients (APIs) from gastrointestinal degradation, and a decrease of gastrointestinal adverse effects (Jebastin K, Narayanasamy D. Rationale utilization of phospholipid excipients: a distinctive tool for progressing state of the art in research of emerging drug carriers. J Liposome Res. 2023 Mar; 33 (1): 1-33).

Use of phospholipids in drug delivery is not new and there are many reports of various drugs successfully formulated into liposomes, mixed micelles, nano- and microemulsions, Self-emulsifying Drug Delivery Systems (SEDDS), Solid Lipid Nanoparticles (SLN), Suspensions, Phospholipid-Drug Complexes, and potentially other molecular and supramolecular complexes (Fricker G, Kromp T. Wendel A, Blume A, Zirkel J, Rebmann H, Setzer C, Quinkert R O, Martin F. Müller-Goymann C. Phospholipids and lipid-based formulations in oral drug delivery. Pharm Res. 2010 Aug; 27 (8): 1469-86). It must be noted that the terminology related to emulsions (micro-, nano-, macro-, and true emulsion) is often confusing and does not necessarily reflect the size of the particles. E.g., the name microemulsion describes surfactant-oil micelles (10-100 nm) solubilized with a co-surfactant (i.e., ionic detergent) and a co-solvent (i.e., short-chain alcohol) (Gutiérrez-Mendez N, Chavez-Garay D R, Leal-Ramos M Y. Lecithins: A comprehensive review of their properties and their use in formulating microemulsions. J Food Biochem. 2022 Jul; 46 (7): c14157). The nanoparticles experimentally detectable in the premix may be micelles and/or liposomes, which upon mixing with Kratom extract and polysorbate, may undergo conversion into mixed micelles and, upon further basification may also form Solid Lipid Nanoparticles. In one aspect of the present teaching, illustrated in Example K, the product is formulated by combining the premix (comprised of lecithin, water, masking agents, sweeteners, and optional polyols, solvents, and sugar alcohols) containing pre-formed nanoparticles and liquid Kratom stock solution (comprised of dry powder Kratom extract solubilized in acid/glycerol/alcohol/water solvent system and Polysorbate 80). Based on this method of production, which does not involve preparation of lipidic film, evaporation steps, sonication, high-pressure homogenization, and the like, the emulsion and nanoparticles may be best described as Self-emulsifying Drug Delivery Systems (SEDDS). Based on the chemical composition and size, it may fit the definition of microemulsion.

The method of preparation presented here consists of the following steps:
1) Preparation of the premix (comprised of lecithin, water, masking agents, sweeteners, and optional polyols, solvents, and sugar alcohols) containing pre-formed nanoparticles.
2) Preparation of the liquid Kratom stock solution (comprised of dry powder Kratom extract solubilized in acid/glycerol/alcohol/water solvent system and Polysorbate 80).
3) Contacting the premix and Kratom stock, and
4) Optionally, adjusting pH of the solution to reduce solubility of mitragynine and other tastants and drive incorporation into the nanoparticles.

Alternatively, as demonstrated in Example M, the premix and the active ingredients may be in the form of dry powder that are blended together in water and allowed to interact to form a palatable drink.

The term “active ingredient” used in this teaching shall be understood as any active principle or payload, pure or not, intended to exert physiological response. Examples include functional nutrition, functional ingredients, functional foods, nutritional supplements, botanicals, extracts, phytochemicals, active pharmaceutical ingredients, oral dosage form drug products, vitamins, minerals, amino acids, etc.

A method was reported for loading polyphenols into nanoliposomes made of soy lecithin (Chen M, Li R, Gao Y, Zheng Y, Liao L, Cao Y, Li J, Zhou W. Encapsulation of Hydrophobic and Low-Soluble Polyphenols into Nanoliposomes by pH-Driven Method: Naringenin and Naringin as Model Compounds. Foods. 2021 Apr. 28; 10 (5): 963). Polyphenols in the publications are more soluble at basic pH, so the method reported in the publication required acidification of the mixture (not basification as with acid soluble Kratom extract). In the present teaching, the preparation of the premix did not involve high pressure homogenization.

In one aspect of the present teaching, a liquid Kratom extract, prepared at pH below 5.0, is neutralized in the presence of phospholipids without the specific goal of formation of liposomes (micelles, mixed micelles, or the like). Instead, any form of particles (in nano- or micro-range, such as Solid Lipid Nanoparticles or phospholipid coated particles), and any suspension (stable or sedimenting, which can be dispersed by shaking), may be generated by following the general approach outlined above where the liquid Kratom extract is combined with a premix (emulsion, dispersion or the like) containing phospholipids, such that it caused pH shift driven precipitation and interaction with phospholipids.

In one aspect of the present teaching, dry powder Kratom extract is suspended, dispersed, or emulsified in the presence of phospholipids and other components of a taste masking system, with the goal of coating the dry powder particles with phospholipids, and producing a suspension (stable or sedimenting, which can be re-dispersed by shaking). This can be achieved by following the general approach outlined above except that instead of the liquid Kratom extract, a fine (micronized) powder of dry Kratom extract is combined with a premix (emulsion, dispersion or the like) containing phospholipids. The formulation, premix, and method of preparation is further described in detail in Example M where L-Leucin, a highly unpalatable and water insoluble active ingredient, is blended with liquid or dry premix to yield a palatable drink.

The relatively low bioavailability of Kratom may be caused by its acid lability, which leads to degradation in gastric juices and effects of first pass metabolism. Delivery of Kratom via oromucosal route (buccal, sublingual) can reduce the efficacious dose and even reduce the onset time. A formulation intended for oromucosal delivery must meet stringent requirements for high palatability but should also contain mucosal epithelium penetration enhancers. Few reports demonstrate that Phospholipids do enhance oromucosal and transdermal delivery (Lankalapalli S. Tenneti V S. Formulation and Evaluation of Rifampicin Liposomes for Buccal Drug Delivery. Curr Drug Deliv. 2016; 13 (7): 1084-1099) (El-Samaligy M S, Afifi N N, Mahmoud E A. Increasing bioavailability of silymarin using a buccal liposomal delivery system: preparation and experimental design investigation. Int J Pharm. 2006 Feb. 3; 308 (1-2): 140-8) (Tian W, Hu Q, Xu Y, Xu Y. Effect of soybean-lecithin as an enhancer of buccal mucosa absorption of insulin. Biomed Mater Eng. 2012; 22 (1-3): 171-8), therefore formulations presented in the present teaching are particularly suited for oromucosal delivery of unpalatable active ingredients. Analgesic, antinociceptive, anxiolytic, anti-hypertensive, heart failure, are just a few examples of the drugs where fast onset of therapeutic action is critical and may be life-saving. Ajmalicine (Raubasine), an alkaloid structurally similar to mitragynine and present in Kratom and other plant extracts, is widely prescribed as anti-hypertensive. Ajmalicine may be formulated for oromucosal delivery using the formulation and method of present teaching as described in Example N.

The aversive taste of certain active pharmaceutical ingredients reduces the acceptance of formulated drugs and hence results in low patient compliance. For this reason, the masking of an aversive taste is one of the aims pursued in the development of drug products. Drug agencies today mandate that medications for children are made palatable or they will not gain approval and marketing authorization.

Kao Chemical Co., Ltd. (Tokyo, Japan) investigated the role of lecithin and individual phospholipids on the suppression of bitter taste of quinine, berberine, promethazine, propranolol, thiamine, brucine, and peptides and reported that phosphatidic acid, phosphatidyl inositol, and fractionated lecithin were able to suppress bitterness of the tested compounds by up to 80% (Katsuragi Y, Mitsui Y, Umeda T, Otsuji K, Yamasawa S, Kurihara K. Basic studies for the practical use of bitterness inhibitors: selective inhibition of bitterness by phospholipids. Pharm Res. 1997 June; 14 (6): 720-4). The company patented, and marketed BMI-40® (Phosphatidic acid) and BMI-60® (a phospholipid cocktail comprised of phosphatidic acid, phosphatidyl inositol, phosphatidyl ethanolamine, and 5% phosphatidyl choline) specifically as bitterness-suppression agents (Katsuragi Y, Yasumasu T, Yamazawa S, Umeda T, Mitsui T. 1997. Materials and methods to decrease bitterness. Japanese Patent No. 2717509).

Phosphatidic acid (BMI-40®) at 1% w/v was further demonstrated to suppress the bitterness of 0.1 mM quinine hydrochloride solution by 81.7%, where 36.1% of the bitterness-depressing effect was found to be due to adsorption, and 45.6% was due to suppression at the receptor site (Nakamura T, Tanigake A, Miyanaga Y, Ogawa T, Akiyoshi T, Matsuyama K, Uchida T. The effect of various substances on the suppression of the bitterness of quinine-human gustatory sensation, binding, and taste sensor studies. Chem Pharm Bull (Tokyo). 2002 Dec; 50 (12): 1589-93). BMI-60® was used to suppress the bitterness of an antibiotic blend (Saito M. Hoshi M, Igarashi A, Ogata H, Edo K. The marked inhibition of the bitter taste of Polymyxin B sulfate and trimethoprim x sulfamethoxazole by flavored BMI-60 in pediatric patients. Biol Pharm Bull. 1999 Sep; 22 (9): 997-8).

In both the early study published by Katsuragi et al., using multiple bitter compounds, and subsequent study using BMI-60® applied to quinine and L-tryptophane (Takagi S, Toko K, Wada K, Ohki T. Quantification of suppression of bitterness using an electronic tongue. J Pharm Sci. 2001 Dec; 90 (12): 2042-8) the researchers emphasized that while bitterness was suppressed, other taste qualities such as saltiness, sweetness, sourness, were not affected. Also, while phosphatidic acid, phosphatidyl inositol, and, to a lesser extent, phosphatidyl ethanolamine exerted bitter masking effect, phosphatidyl choline was not effective.

Examples B, D. L, and others, demonstrated that formulations of the present teaching, which incorporate lecithins and, optionally phosphatidic acid and phosphatidyl serine, along with sweetener system, and masking agents were efficient at masking not only bitterness, but also saltiness, sourness, and a whole host of other off-flavors and irritants found in Kratom, hemp, other botanical extracts, pea protein, minerals, amino acids, etc.

Testing of various lecithins and individual phospholipids also determined that phosphatidyl choline was less effective at masking the bitterness and other off-flavors of Kratom (Example L). Phosphatidyl serine has never been evaluated as a bitter masker or taste masker. The experiments determined that phosphatidyl serine was a very efficient taste masker of Kratom (Example L). A typical content of phosphatidylserine in commercially available lecithins is less than 3%; therefore, rather than producing phosphatidyl serine by fractionation of lecithin, most commercial preparations of phosphatidyl serine sold as food ingredients or dietary supplements today are produced by enzymatic replacement, using Phospholipase D, of choline group in phosphatidyl choline with serine (Arora H, Culler M D, Decker E A. Production of a High-Phosphatidylserine Lecithin That Synergistically Inhibits Lipid Oxidation with α-Tocopherol in Oil-in-Water Emulsions. Foods. 2022 Mar. 30; 11 (7): 1014).

Liquid and de-oiled lecithins and preparations enriched for specific phospholipids, particularly phosphatidyl serine and phosphatidic acid, are sold as nutritional supplements.

Scientific and clinical evidence accumulated in the past 20 years demonstrates that dietary intake of products containing phospholipids, and especially phosphatidyl serine, has many health benefits, including stress management, improved mood, cognition, and physical performance. Some data also suggest that phospholipids have anti-inflammatory and other protective properties.

A large number of research and clinical trials demonstrated that consumption of 200-900 mg of phosphatidyl serine per day helped fight stress and improved mood, focus, memory, and cognitive functions (Benton D, Donohoe R T, Sillance B, Nabb S. 2001 The influence of phosphatidylserine supplementation on mood and heart rate when faced with an acute stressor. Nutr Neurosci. 4 (3): 169-78.; Baumeister J, Barthel T, Geiss K R, Weiss M. 2008 Influence of phosphatidylserine on cognitive performance and cortical activity after induced stress. Nutr Neurosci. 11 (3): 103-10.; Boyle N B, Dye L, Arkbåge K, Thorell L, Frederiksen P, Croden F, Lawton C 2019 Effects of milk-based phospholipids on cognitive performance and subjective responses to psychosocial stress: A randomized, double-blind, placebo-controlled trial in high-perfectionist men. Nutrition, 57, 183-193.; More M I, Freitas U, Rutenberg D. 2014 Positive effects of soy lecithin-derived phosphatidylserine plus phosphatidic acid on memory, cognition, daily functioning, and mood in elderly patients with Alzheimer's disease and dementia. Advanced Therapeutics, 31 (12): 1247-62.; Hellhammer J, Waladkhani A R, Hero T & Buss C 2010 Effects of milk phospholipid on memory and psychological stress response. British Food Journal, 112, 1124-1137.; Parker A G, Gordon J, Thornton A, Byars A, Lubker J, Bartlett M, Byrd M. Oliver J, Simbo S, Rasmussen C, Greenwood M, Kreider R B. 2011 The effects of IQPLUS Focus on cognitive function, mood and endocrine response before and following acute exercise. J Int Soc Sports Nutr. 21; 8:16). One clinical study demonstrated reduced symptoms of premenstrual syndrome (Schmidt K, Weber N, Steiner M, Meyer N, Dubberke A, Rutenberg D, Hellhammer J. 2018 A lecithin phosphatidylserine and phosphatidic acid complex (PAS) reduces symptoms of the premenstrual syndrome (PMS): Results of a randomized, placebo-controlled, double-blind clinical trial. Clin Nutr ESPEN. 24:22-30). The responses were potentially mediated through a modulation of endocrine responses, including cortisol availability and increased testosterone/cortisol ratio (Schubert M, Contreras C, Franz N, Hellhammer J (2011): Milk-based phospholipids increase morning cortisol availability and improve memory in chronically stressed men. Nutrition Research, 31, 413-420.; Hellhammer J, Fries E, Buss C, Engert V, Tuch A, Rutenberg D, Hellhammer D. 2004 Effects of soy lecithin phosphatidic acid and phosphatidylserine complex (PAS) on the endocrine and psychological responses to mental stress. Stress. 7 (2): 119-26.; Hellhammer J, Vogt D, Franz N, Freitas U, Rutenberg D. 2014 A soy-based phosphatidylserine/phosphatidic acid complex (PAS) normalizes the stress reactivity of hypothalamus-pituitary-adrenal-axis in chronically stressed male subjects: a randomized, placebo-controlled study. Lipids Health Dis. 13:121.; Starks M A, Starks S L, Kingsley M. Purpura M. Jager R. 2008 The effects of phosphatidylserine on endocrine response to moderate intensity exercise. J Int Soc Sports Nutr. 28; 5:11.; Contarini G, Povolo M 2013 Phospholipids in milk fat: Composition, biological and technological significance, and analytical strategies. International Journal of Molecular Sciences, 14, 2808-2831).

Several trials have shown the significant benefits of milk phospholipids on cognitive outcomes. These benefits are well established in infants, where dairy ingredients that contain milk phospholipids can be added to infant formulas to match human breast milk more closely. Both animal and human trials have shown benefits for the improvement in cognitive development in infants (Ambrożej D, Dumycz K, Dziechciarz P, Ruszczyński M. 2021 Milk Fat Globule Membrane Supplementation in Children: Systematic Review with Meta-Analysis. Nutrients. 13 (3): 714.; Gurnida D A, Rowan A M, Idjradinata P, Muchtadi D, Sekarwana N 2012; Association of complex lipids containing gangliosides with cognitive development of 6-month-old infants. Early Human Development, 88, 595-601; Timby N, Domellof E, Hernell O, Lönnerdal B, Domellöf M 2014 Neurodevelopment, nutrition, and growth until 12 mo of age in infants fed a low energy, low-protein formula supplemented with bovine milk fat globule membranes: A randomized controlled trial. American Journal of Clinical Nutrition, 99, 860-868.). Some data demonstrate similar effects in adults and the elderly (Gallier S, MacGibbon A K. McJarrow P 2018 Milk fat globule membrane (MFGM) supplementation and cognition. Agro Food Industry Hi-Tech, 29, 14-16.; Richter Y, Herzog Y, Lifshitz Y, Hayun R, Zchut S. 2013 The effect of soybean-derived phosphatidylserine on cognitive performance in elderly with subjective memory complaints: a pilot study. Clin Interv Aging. 8:557-63).

Inflammation underlies many adverse health outcomes, and anti-inflammatory foods and diets have been widely popularized in recent years. Both in vitro and in vivo work indicate that both phospholipid-rich and ganglioside-rich components impact a range of anti-inflammatory pathways (Palmano K P. MacGibbon A K H, Gunn C A. Schollum L M. 2020 In Vitro and In Vivo Anti-inflammatory Activity of Bovine Milkfat Globule (MFGM)-derived Complex Lipid Fractions. Nutrients. 12 (7): 2089.; Park E J. Suh M. Thomson B. Ma D W L, Ramanujam K. Thomson A B R. Clandinin M T 2007 Dietary ganglioside inhibits acute inflammatory signals in intestinal mucosa and blood induced by systemic inflammation of Escherichia coli lipopolysaccharide. Shock, 28, 112-117). Numerous scientific reports support the anti-inflammatory properties of milk phospholipids in humans (Dalbeth N, Ames R, Gamble G D, Horne A, Wong S. Kuhn-Sherlock B, MacGibbon A, McQueen F M. Reid I R & Palmano K 2012 Effects of skim milk powder enriched with glycomacropeptide and G600 milk fat extract on frequency of gout flares: a proof-of-concept randomised controlled trial. Annals of the Rheumatic Diseases, 71, 929-934.; Zanabria R. Tellez A M, Griffiths M, Sharif S, Corredig M. (2014) Modulation of immune function by milk fat globule membrane isolates. J Dairy Sci. 97 (4): 2017-26.; Demmer E, Van Loan M D, Rivera N, Rogers T S, Gertz, E R, Bruce German J, Smilowitz J T. Zivkovic A M, German J B, Smilowitz J T, Zivkovic A M, 2016 Addition of a dairy fraction rich in milk fat globule membrane to a high-saturated fat meal reduces the postprandial insulinaemic and inflammatory response in overweight and obese adults. Journal of Nutritional Science, 5, 1-11). One recent animal study demonstrated the cardioprotective effect of phosphatidyl serine, with both cytoprotective and anti-inflammatory mechanisms involved (Schumacher D. Curaj A, Staudt M, Cordes F. Dumitrascu A R, Rolles B. Beckers C. Soppert J. Rusu M, Simsekyilmaz S. Kneizch K, Ramachandra C J A, Hausenloy D J, Lichn E A. 2021 Phosphatidylserine Supplementation as a Novel Strategy for Reducing Myocardial Infarct Size and Preventing Adverse Left Ventricular Remodeling. Int J Mol Sci. 22 (9): 4401).

In one aspect of the present teaching that phospholipids act not only as taste masking agents and excipients (co-solvents, emulsifiers, penetrants, complexation agents, bioavailability enhancers, etc.) but also as nutritional supplements with their own health benefits which, in some cases, may act in additive or synergistic manner with the unpalatable active ingredients.

Despite the fact that lecithin/phospholipids were first reported to mask bitterness in 1997 (Katsuragi Y, Mitsui Y, Umeda T, Otsuji K, Yamasawa S, Kurihara K. Basic studies for the practical use of bitterness inhibitors: selective inhibition of bitterness by phospholipids. Pharm Res. 1997 June; 14 (6): 720-4) and commercial preparations specifically developed for that purpose were available for at least 20 years, there are very few literature reports on the use of lecithins or phospholipids specifically for taste masking purposes outside of strictly pharmaceuticals (Zheng X, Wu F, Hong Y, Shen L, Lin X. Feng Y. Developments in Taste-Masking Techniques for Traditional Chinese Medicines. Pharmaceutics. 2018 Sep. 12; 10 (3): 157). In one case phospholipids were used for the purpose of improved bioavailability of solid formulations and reduction of off-flavors came as a welcome bonus (Chuah A M, Jacob B, Jie Z, Ramesh S, Mandal S, Puthan J K, Deshpande P, Vaidyanathan V V, Gelling R W, Patel G, Das T, Shrecram S. Enhanced bioavailability and bioefficacy of an amorphous solid dispersion of curcumin. Food Chem. 2014 Aug. 1; 156:227-33).

Bitter maskers/blockers, and other off-flavor maskers are a standard part of drinkable and eatable foods and supplements. Many of the energy and sports drinks that use specific minerals, vitamins, and botanicals have to use the maskers to achieve palatability. Use of standard commercially available masker(s) is usually sufficient to mask unpalatable active ingredients such as minerals, vitamins, amino acids, and their concentrations used in a typical energy drink. Formulating botanicals in their concentrated forms appears to be a significantly more difficult masking challenge because botanical extracts are all multicomponent, complicated systems, in which both the aversive components and the structure-activity relationship are generally unclear. Thus, it is very difficult to discover a universal taste-masking technique to cover the aversive taste of such systems, and there are also relatively limited studies paying attention to this field (Zheng X, Wu F, Hong Y, Shen L, Lin X, Feng Y. Developments in Taste-Masking Techniques for Traditional Chinese Medicines. Pharmaceutics. 2018 Sep. 12; 10 (3): 157).

Most of the phytochemical work performed on Kratom was dedicated to mitragynine and related indole alkaloids therefore other phytochemicals that coextract and copurify with mitragynine are not well studied. One report mentions flavonoids, triterpenoids, saponins, and glycosides, all known to be bitter (Raffa, R. B. (Ed.) Kratom and Other Mitragynines: The Chemistry and Pharmacology of Opioids from a Non-opium Source; CRC Press: New York, NY, USA, 2015). One other report identified Chlorogenic acid, Umbelliferone, O-coumaric acid, Quercetin, Quercetin 3-galactoside 7-rhamnoside, Rutin, Isoquercitrin, Vincamine, and alpha-linolenic acid (Goh Y S, Karunakaran T. Murugaiyah V, Santhanam R, Abu Bakar M H, Ramanathan S. Accelerated Solvent Extractions (ASE) of Mitragyna speciosa Korth. (Kratom) Leaves: Evaluation of Its Cytotoxicity and Antinociceptive Activity. Molecules. 2021 Jun. 17; 26 (12): 3704). Most, if not all, of these compounds are known to carry aversive tastes. Chlorogenic acid, a polyphenol abundant in coffee, is known to affect astringency, sweetness, bitterness, metallic, and sour tastes. Its degradation products (caffeic and quinic acids) make up 60-70% of the bitter taste in coffee (Kunisuke Izawa, Amino Y, Kohmura M, Ueda Y, Kuroda M, Human-Environment Interactions-Taste, Editor(s): Hung-Wen (Ben) Liu, Lew Mander, Comprehensive Natural Products II, Elsevier, 2010, Pages 631-671, ISBN 9780080453828). Both coffee and M. speciosa belong to Rubiaceae family and Kratom extraction involves evaporation under elevated temperatures, therefore some bitterants present in roasted coffee (caffeic and quinic acids and potentially others) are expected to be present in Kratom extracts. Alpha-linolenic acid, an omega-3 fatty acid, is another compound identified in Kratom extracts that carries bitter, fishy, rancid, and other off notes. Umbelliferone and O-coumaric acid are coumarines with bitter, sour, aromatic, and astringent off-flavors. Rutin and Quercetin are bitter flavanones.

Bitter maskers are used in many marketed Kratom shots, but they provide a very limited improvement. In the present teaching, lecithin/phospholipids were utilized to block off-flavors. Another aspect of the present teaching is that lecithin and phospholipids were not only effective bitter blockers, but efficiently reduced irritation and other off-flavors, and dramatically improved the mouthfeel. The use of lecithin and phosphatidyl serine suppressed not only off-flavors but in some cases also suppressed the added flavors. This is fixed by adding higher concentrations of the flavors, flavor enhancers, and careful pairing of flavors with the base. In another aspect of the present teaching, lecithins at concentrations that high will impart its own off-flavors and even bitterness. Liquid lecithins generally will carry more off-flavors than de-oiled lecithins, which may be explained by the presence of free fatty acids in liquid lecithins (Stephan, A., Steinhart, H. (2000) Bitter taste of unsaturated free fatty acids in emulsions: contribution to the off-flavor of soybean lecithins. Eur Food Res Technol 212, 17-25). The taste of lecithin is generally described as strawy, roasty, nutty, or haylike. Therefore, selection of taste maskers that will be effective at both. (1) complementing the action of lecithin in masking the botanicals and (2) masking the lecithin itself was also a more difficult masking challenge than usual.

The current teaching (Examples K, L, M) demonstrates that phosphatidyl serine and phosphatidic acid may be used as alternatives to crude lecithins or combined with liquid or de-oiled lecithins to improve the formulations' organoleptic properties. Phosphatidyl serine and phosphatidic acid, on their own, were efficient maskers of Kratom and carried fewer off-flavors. However, the use of purified phospholipids may be economically prohibitive. Furthermore, unlike lecithins, which are mostly phosphatidyl choline, the other phospholipids including phosphatidyl serine and phosphatidic acid, are not easily dispersible in aqueous solutions. Current teaching provides methods of incorporation of phosphatidyl serine and phosphatidic acid into predominantly lecithin-based formulations to achieve desired product characteristics.

The combination of de-oiled lecithin and liquid lecithin may also be a factor affecting emulsion stability. Under some manufacturing protocols, and in some formulation matrices, high concentration of oils, contained in liquid lecithin, may lead to oil phase separation. To stabilize the emulsion, oil content may be reduced by reducing the amount of liquid lecithin used and adding a de-oiled lecithin. If higher concentrations of oil will be necessary to maintain the mouthfeel, taste masking, or entrapment efficiency, bound forms of oils may be used, as taught in Example M. Example of such oils are Nutri Sperse HOS 70ND, a powder form of high oleic oil from sunflower, Nutri Sperse MCT 70, a powder form of medium chain tricarboxylic acids (Abitec, 501 West 1 Ave, Columbus, OH 43215) and many others available on the market today.

It is generally known that sweet taste can efficiently suppress bitterness; however, Kratom shots and similar botanical extracts are typically too bitter and have too many other aversive flavors to rely on sweetness alone as a taste masker. The present experiments demonstrated that saturated solutions of sucrose (52% w/v) were not able to mask bitterness, other off-flavors, and irritation caused by Kratom extracts. The present experiments also demonstrated that a high level of sweetness worked in concert with phospholipids and other maskers and significantly contributed to masking the bitterness, other off-flavors and irritation of Kratom, hemp, other botanicals, minerals, amino acids, proteins and pharmaceuticals tested in the present teaching. Without the potent sweetener systems of the present teaching, the same levels of palatability will be impossible to achieve; therefore, a sweetener system, as described in detail below, is thus another aspect of the present teaching.

Sucrose (table sugar) is one of the common sweeteners in all taste masking applications due to its natural round sweetness, good mouthfeel, and low cost. However, it may not be enough to use sucrose alone to suppress the bitterness of Kratom extracts. The present experiments determined that at higher concentrations of the product (>7 mg/ml) most of the lower purity extracts (<70%) required an additional source of sweetness, typically a high intensity sweetener (HIS), a sweetness enhancer, or a taste modulator/modifier. In some aspects, a formulation contains all four components of the sweetener system.

Sucrose is calorigenic and cariogenic, causes strong glycemic response, and must be avoided by diabetics. High intensity sweeteners (HIS), sometimes combined with low intensity sweeteners (LIS) both caloric (e.g., sucrose, fructose, tagatose) and non-caloric (e.g., crythritol, allulose, sorbitol, xylitol, sorbose, allose, etc.) are commonly used to formulate sugar free or reduced sugar foods, supplements, and pharmaceuticals. Most HIS will carry off-tastes of their own (most frequently metallic, chemical, or bitter) and must be carefully selected and blended with other HIS and LIS to minimize off-flavors and maximize sweetness. The present experiments determined that some HIS enhanced irritation and off-flavors caused by Kratom extracts. Sucralose and stevia, present in some commercial Kratom products, enhanced irritation and tingling. In one aspect of the present teaching, the composition is completely devoid of sucralose and stevia.

One other aspect of the present teaching is that liquid Kratom has a very fast taste release (onset) and a strong lingering aftertaste. The sweetener system, then, has a similarly fast sweetness release and a sweet lingering component to help mask the aftertaste, as demonstrated in multiple examples.

In one aspect of the present teaching, a sugar free Premix formula is comprised of:
Glycerol—10% v/v
Masking agent OSF 2070C—0.4% w/v
Masking agent MET 6985—2% w/v
Allulose—20% w/v
Sodium saccharin—0.05% w/v
Neohesperidin dihydrochalcone—0.025% w/v
Monk fruit extract, 25% mogroside V—0.15% w/v
Water

Saccharin sodium (or Aspartame, Acesulfame K, Neotame, or other fast acting sweeteners known to formulators) acts as fast sweetener. Neohesperidin dihydrochalcone acts as a lingering sweetener, bitter masker, and taste modulator. Monk fruit extract acts as sweetener, taste modulator, and mouthfeel enhancer. Glycerol and Allulose (or Xylitol, Erythritol, Sorbitol, or other LIS) act as mouthfeel enhancers, reduce irritation, add less than 25% of sweetness, yet help create round sweetness. The sweeteners, their combinations, concentrations, and ratios in the present teaching were optimized in multiple screens, however other combinations may be envisioned based on the data provided here.

Neohesperidin dihydrochalchone contributes to masking Kratom and other unpalatable active ingredients used in the present teaching but also helps reduce off-flavors of lecithins. This sweetener with taste modulating and bitter masking properties belongs to flavanone glycosides (natural flavonoids) which include both sweet and bitter members which are structurally related (Frydman A, Liberman R, Huhman D V, Carmeli-Weissberg M, Sapir-Mir M, Ophir R, W Sumner L, Eyal Y. The molecular and enzymatic basis of bitter/non-bitter flavor of citrus fruit: evolution of branch-forming rhamnosyltransferases under domestication. Plant J. 2013 Jan; 73 (1): 166-78). The natural flavonoids naringin and neohesperidin are bitter, but their dihydrochalcone derivatives become intensely sweet. Neohesperetine is mildly sweet but also acts as a sweet enhancer. Structurally close Eriodictyol and homoeriodictyol are potent bitter maskers. Neohesperidin dihydrochalcone has slow onset and a lingering aftertaste not desirable in some products, therefore numerous compounds have been synthesized by modifying neohesperidin dihydrochalcone or aglycone hesperetin dihydrochalcone. Phyllodulcin, a natural dihydroisocoumarin, is also an intensely sweet compound possessing taste properties similar to those of dihydrochalcone. Dihydroisocoumarin and dihydrochalcone have structural similarities enabling an insight into structure function relationships (Shin W, Kim S J, Shin J M, Kim S H. Structure-taste correlations in sweet dihydrochalcone, sweet dihydroisocoumarin, and bitter flavone compounds. J Med Chem. 1995 Oct. 13; 38 (21): 4325-31).

The general structural relationship and taste modulating properties of flavanones and coumarins (both of which were copurified with mitragynine during Kratom extraction; Goh Y S, Karunakaran T, Murugaiyah V, Santhanam R, Abu Bakar M H, Ramanathan S. Accelerated Solvent Extractions (ASE) of Mitragyna speciosa Korth. (Kratom) Leaves: Evaluation of Its Cytotoxicity and Antinociceptive Activity. Molecules. 2021 Jun. 17; 26 (12): 3704) suggest an overlap in the receptors they interact with, and a strong potential for the flavanones with sweet taste, taste modulation, and bitter masking properties to be efficient Kratom maskers and synergists with phospholipids. Neither neohesperidin dihydrochalchone, nor related sweeteners, are listed in ingredient lists of any Kratom, hemp, or other botanical formulations. Use of these compounds is therefore an aspect of the present teaching. The non-comprehensive list includes criodictyol, homocriodictyol, hesperetin, neohesperidin dihydrochalcone, naringenin, naringin dyhydrochalcone, and other flavanones and flavanone glycosides and phyllodulcin.

Neohesperidin dihydrochalcone was procured from Bordas (Calle Acueducto N° 4-6, Polígono Industrial La Isla 41703, Dos Hermanas, Sevilla, Spain) or from M&U International (31 Readington Rd., Branchburg, NJ 08876, USA). Other Neohesperidin derivatives, such as Citrosa+specifically developed for taste masking and sweetening purposes (HTBA, Avinguda Diagonal, 567 4th Fl., Barcelona, Spain 08029). The compound is known to have low water solubility. Therefore, in the liquid formulations of the present teaching neohesperidin dihydrochalcone was used as a 10% w/v stock solution in propylene glycol. Preparation of such propylene glycol stock, which is stable for weeks, or a hot water stock, which is stable only for a few hours before it precipitates, is a standard industry practice, and other methods to improve solubility in use have been proposed (Benavente-García O, Castillo J, Del Baño MJ, Lorente J. Improved water solubility of neohesperidin dihydrochalcone in sweetener blends. J Agric Food Chem. 2001 Jan; 49 (1): 189-91).

A perception of sweetness can also be achieved without any sweeteners via taste modulation as in the case of use of Synsepalum dulcificum (“miracle berries”) and the protein miraculin responsible for the taste modulation effect. Miraculin itself does not taste sweet but it interacts with receptors in such a way that sour-tasting acidic foods, such as citrus, are perceived as sweet and the effect can last for up to two hours. A number of scientific studies have been reported where miraculin was incorporated into foods or specialized diets (Rodrigues J F, Andrade R D S, Bastos S C, Coelho S B, Pinheiro A C M. Miracle fruit: An alternative sugar substitute in sour beverages. Appetite. 2016 Dec. 1; 107:645-653). Lyophilized berries of S. dulcificum or tablets made of berry powder are widely available. In one aspect of the present teaching S. dulcificum berry, its preparations or extracts, or miraculin, or its recombinantly produced analogs, is used to impart sweetness and mask the aversive sour/acidic taste of Kratom. As illustrated in Example Q, consuming miracle berry prior to ingestion of an unsweetened Kratom shot, converted its sour/acidic taste into sweetness. Other taste modulators, with similar functionality e.g., curculin protein from Curculigo latifolia may be used. In another aspect of the present teaching, other botanical extracts with sour/acidic notes, e.g., Bacognize® and LJ100®, and other unpalatable active ingredients with sour/acidic notes, e.g., citrulline malate, may be formulated without sweeteners.

Both commercial masking agents OSF 2070C and MET 6985 are proprietary blends developed by OSF Flavors and Foodarom, respectively. The manufacturers revealed that both preparations incorporate sweetness enhancing compounds of undisclosed botanical origin. It is therefore another aspect of the present teaching.

As known to those skilled in art of formulation and flavoring, commercially available preparations of flavors, taste maskers (or masking agents), bitter maskers (or bitter blockers), mouthfeel enhancers, taste and flavor enhancers, etc., especially of natural origin, such as ones used in the present teaching, are highly complex products that are proprietary to the manufacturer (Flavor House). The flavors and maskers referenced in the present teaching are attributable to their manufacturers based on the first three letters of the code. Products starting with OSF are sold by OSF Flavors, (OSF Flavors, 40 Baker Hollow Rd, Windsor, CT 06095), and products starting with MET are sold by Foodarom-Glanbia Nutritionals (Foodarom, 5400 Rue Armand-Frappier, Saint-Hubert, QC J3Z 1G5, Canada). These products are commercially available by referencing the product code. The same maskers, flavors, enhancers, used in the Examples below or their functional equivalents, from the same or different flavor manufacturers may be used when the present teaching is practiced.

In another aspect of the present teaching, a formula contains de-oiled lecithin as the source of phospholipids and a high concentration of Allulose (or sucrose or other sugar) is used in combination with specific co-solvents to create a transparent formula with visual appeal. Allulose at 56% was used in one formulation with specific Kratom extract and flavor. Other LIS, both caloric (sugar, fructose, and others) and non-caloric (Xylitol, Sorbitol, and others), or their combinations may be used in other aspects of the present teaching. Erythritol may also be incorporated by combining with others, for its mouthfeel and cooling effect, albeit at lower percentages due to its relatively low solubility.

Phospholipids used for formulation of the present teaching may be purified, partially purified, or be in the form of lecithin, milk phospholipids (phospholipids in milk fat globule), or other forms.

### Specific Examples Include

1) Liquid lecithin, e.g., containing oils and derived from soy, sunflower, egg yolk and other sources.
2) De-oiled lecithin, e.g., dry powders derived from soy, sunflower, egg yolk and other sources. Supplied by Vivion, Inc (929 Branstein Road, San Carlos, CA 94070) as soy lecithin powder or by Quality Supplements and Vitamins, Inc, (Ft Lauderdale, FL 33309) as granules.
3) Milk phospholipids (phospholipids in milk fat globule) derived from butterscotch and other products derived from processing of dairy products, e.g., those supplied by Fonterra, Inc.
4) Purified phospholipids, especially phosphatidyl choline, including its chemical derivatives, primarily used for their liposome forming properties, as dry powder (Lipoid H 50) or dissolved in oil (Phosal H 50), as supplied by American Lecithin Company, LLC or Lipoid GMBH, Ludwigshafen, Germany or Chemi Nutra, LLC, (11100 Metric Blvd., 200D, Austin, TX 78758), or Lipoid, or ECA, Healthcare (1260 E. Locust st. Ontario, CA 91761, USA).
5) Purified phospholipids, especially phosphatidic acid, phosphatidyl serine, phosphatidyl inositol, phosphatidyl ethanolamine, and others, including chemical derivatives, primarily used for their taste masking effect, as supplied by Chemi Nutra, LLC, ECA Healthcare, or Lipoid.
6) Various blends of lecithins and/or phospholipids that may serve either liposome forming or taste masking purposes, or both, commercially available, as supplied by Kao corporation, or specifically formulated for Kratom, other botanicals, pharmaceuticals and the like.

Various preparations of lecithin can be used at 0.5-5%, 0.5-10%, 0.5-20%, or 0.5-30% of the final concentration of the formulation, while purified or partially purified phospholipids, e.g., phosphatidyl choline, can be used at concentrations below 0.5%. The final concentration of phospholipids is determined by the concentration and nature of the tastants, in the product and the degree to which aversive taste is reduced in order to achieve palatability of the product.

In another aspect of the present teaching, the formulation is stable and does not show signs of physical separation of phases for at least 12 months. In another aspect of the present teaching, the formulation separates into distinct phases, e.g., lipid layer on top, aqueous or nanoemulsion phase in the middle, and particles at the bottom. In some formulations particles are of lipophilic nature and, instead of settling at the bottom, will colocalize at the top with the lipids. Sometimes particles are of high density and will settle at the bottom and pull the lipids with them (for example when Aloe vera preparation is incorporated, Example O). Formulations separate over time and form distinct phases such as lipid, aqueous, phospholipid, solid/insoluble pellet, and can be briefly agitated prior to consumption.

Formulations prepared with liquid lecithins are opalescent to opaque. The higher the concentration of lecithin the more opaque the appearance. High concentrations of polyols, sugars, or sugar alcohols, e.g., sucrose or erythritol, turn formulations opalescent and translucent. Formulations prepared with de-oiled lecithins and high concentrations of polyols, sugars, or sugar alcohols are translucent and, when placed in a clear glass or plastic vessel of 10-100 ml, like the one that may be used for delivery of one to five servings of Kratom, appears transparent. Transparent formulations are particularly visually appealing to the end consumer as they are perceived clear and delicious. Therefore, in another aspect of the present teaching, the formulation is not only palatable but also transparent or slightly opalescent.

In the context of the present teaching, “transparent” shall be defined as having optical density of less than approximately 0.2 at 600 nm, when measured as described in Example K. Generally speaking, transparent may not be completely clear and may have turbidity (opalescence), and when placed in a glass vessel with 1-3 cm liquid layer, a regular text can be read through it. Translucent is a sample that is not transparent, yet allows the light through and has an OD₆₀₀ between 0.3 and 0.9. Opaque is a sample that does not allow the light through under the same conditions.

The other aspect of Kratom formulation is viscosity. The higher the concentrations of lecithin in the formulation, the higher its viscosity. Solutions containing liquid lecithins will have higher viscosity than solutions containing de-oiled lecithins at the same concentration.

High concentrations of polyols, sugars, sugar alcohols will also result in high viscosity.

Some viscosity may be desirable because it helps create the right mouthfeel and improves overall organoleptic profile. In some cases, viscosity is known to aid taste masking, potentially due to limiting the interaction between the tastants and receptors. Excessive viscosity, on the other hand, reduces efficiency of mixing and pumping the solutions (machinability), increases production time and therefore complicates the manufacturing process. With higher concentrations of sugars, sugar alcohols, Liquid Kratom shots are typically sold in 10-60 ml bottles as 1-3 servings per bottle. Excessive viscosity will delay bottle emptying, complicate splitting of the content into individual servings, lead to excessive amount of liquid lost on the bottle walls resulting in product waste and underdosing.

As discussed in detail above, sweetness is one of the parameters in taste masking of Kratom and thus very high concentrations of sucrose may be required to achieve optimal palatability. Replacing sugar with HIS is possible; however, to achieve natural “round” sweetness of sucrose HIS are typically blended with non-caloric Low Intensity Sweeteners (LIS), e.g., erythritol, allulose, sorbitol, etc. It is therefore desirable that viscosity of the formulation is optimized/balanced between organoleptic properties vs machinability and dosing convenience/efficiency.

For sugar free formulations containing 6% de-oiled lecithin the concentration of LIS used was between about 20% (in one example, 10% glycerol and 10% allulose or erythritol) and about 40% (in one example, 20% glycerol and 20% allulose or erythritol).

One report of transparent formulation containing lecithin is presented in U.S. Pat. No. 10,898,873. The formulations are comprised of sugar or sugar alcohol, alcohol, cosurfactant (e.g., Polysorbate), water, oil, and lecithin. However, the formulations were primarily intended for improved oil separation, not intended for consumption and were not optimized for organoleptic properties and no examples of the use for taste masking purposes were provided. In one aspect of the present teaching, the formulation contains no oil.

In another aspect of the present teaching, the formulation is transparent. The formulation was developed to:
a) maintain transparent appearance in the presence of high concentrations of Kratom extract,
b) off-flavors) have good overall palatability,
c) use only food ingredients or approved food additives not exceeding their Acceptable Daily Intake quantities,
d) remain stable upon storage without precipitation or phase separation.

Several iterations of screening and optimization were conducted. One representative experiment is presented in EXAMPLE H.

In another aspect of the present teaching, a transparent formulation is prepared by making premix “Transparent 53” comprised of:
De—oiled lecithin—4% w/v
Glycerol—10% v/v
Propylene glycol—7.5% v/v
Ethanol—12.5% v/v
Masking agent OSF 7186B—0.5% w/v
Masking agent OSF 2070C—0.4% w/v
Masking agent MET 6985—2% w/v
Allulose—56% w/v
Sodium saccharin—0.025% w/v
Monk fruit extract, 25% mogroside V—0.15% w/v
Water—q.s. 1000 ml

And by mixing the premix with liquid stock solution of Kratom, Example K.

De-oiled lecithin and purified phospholipids are generally chosen over crude liquid lecithins as they possess less of their own flavors and produce formulations with cleaner organoleptic profile. Liquid lecithins may be used due to their cost advantages and delivery of oils which contribute to taste masking and mouthfeel (Example K), or clean tasting oils may be added (Example M).

Alternatively, the formulation can be stabilized by optimization of water/phospholipid ratio, water/oil/phospholipid ratio, addition of co-solubilizers, surfactants, emulsifiers, whey protein or casein, dietary fiber, viscosity modifying agents, silica, manipulation of density, and other methods known to those skilled in art of formulation.

Polysorbate 80 used in the formulations of the present teaching is one in the broad selection of co-solubilizers (surfactants or emulsifiers) widely used in foods, supplements, and pharmaceuticals. Other compounds with similar functionality, approved for food and pharmaceutical applications, can be used and may produce better results depending on the specific formula. Examples of such surfactants and emulsifiers are: other polysorbates, sorbitan esters, polyglyceril esters, sucrose esters, alkyl polyglucosides, poloxamers, an ethylene oxide/propylene oxide block copolymers, ethoxylated fatty acids, ethoxylated monoglycerides, sodium lauryl sulfate, quillaja, rhamnolipids, sophorolipids, and others, as well as their combinations.

### EXAMPLES

| Example | Description |
|---|---|
| A | Various percentages of purity from pure Kratom vs taste |
| B | Kratom maskers and flavors |
| C | Titration of PS 80 for stabilization |
| D | Hemp maskers and flavors |
| E | Bacognize, LJ100, and mushroom extracts |
| F | Caffeine and antibiotics |
| G | Effect of citrate vs phosphate on irritation |
| H | Development of transparent formulation |
| I | Lecithin concentration vs palatability. |
| J | pH of various commercial products |
| K | Solubility and entrapment efficiency vs pH. Organoleptic |
| | and physico-chemical properties of various formulations. |
| L | Use of individual purified phospholipids and combinations |
| | with lecithin |
| M | Liquid and dry powder premix for masking of a homemade |
| | sports nutrition drink |
| N | Buccal delivery of ajmalicin |
| O | Non-stable suspension |
| P | Solid formulation, Kratom protein bar |
| Q | Use of taste modulator Miracle Berry Synsepalum dulcificum |
| | in place of sweeteners |

### Example A

Dry powder Kratom extracts from Pure Kratom, LLC (1931 West Bay Dr., Largo, FL 33770).

To prepare liquid Kratom stock solutions four individual samples, each corresponding to mitragynine content of 45%, 55%, 70%, and 90% w/w, were dissolved in EGP solvent at a final concentration of mitragynine 60 mg/ml each. Samples were heated to 70° C. to facilitate dissolution.

### EGP Solvent

Ethanol—18% v/v
Glycerol—45% v/v
Phosphoric acid—1.22% (q.s to pH 3.8)
Water—35% v/v (q.s. to volume)

The 60 mg/ml Kratom stock solutions were immediately diluted to 10 mg/ml in distilled water. Upon dilution, the 45% sample developed fine precipitate whereas other samples remained fully soluble and transparent. The diluted samples were immediately subjected to organoleptic evaluation by two panelists trained to identify Kratom off-flavors. Each parameter was rated on a scale from 0 to 10, with 0 corresponding to undetectable and 10 corresponding to the level detectable in the 45% sample.

TABLE A1
Off-flavors vs. mitragynine percent in dry powder extract.

| | 90% | 70% | 55% | 45% |
|---|---|---|---|---|
| | extract | extract | extract | extract |
| bitterness | 3 | 6 | 7 | 10 |
| herbal, dry leaves | 1 | 3 | 8 | 10 |
| earthy | 1 | 4 | 7 | 10 |
| pucky/astringent | 4 | 7 | 10 | 10 |
| sour/acidic | 3 | 6 | 7 | 10 |
| lingering bitterness | 4 | 7 | 10 | 10 |
| irritation and tingling | 2 | 4 | 7 | 10 |

### Example B

This Example demonstrates that there is a tremendous variety of bitter maskers and taste maskers offered by flavor houses but most of them are powerless to provide a meaningful reduction of off-flavors in a Kratom shot. A vast and meticulous screen of maskers had to be conducted to identify the three to be combined with each other and with lecithin and with a powerful sweetener system to deliver a palatable formulation.

Liquid Kratom stock solution was prepared using dry powder extract with mitragynine content 70% (from Pure Kratom, LLC). The powder was dispersed in EGC solvent at final concentration of mitragynine at 80 mg/ml. Heated to 70° C. to facilitate dissolution. Polysorbate 80 was added to a final concentration of 5.5%.

### EGC Solvent

Ethanol—18% v/v
Glycerol—45% v/v
Citric acid monohydrate-5% w/v (pH 3.8)
Water—35% v/v (q.s. to volume)

Premix (2% or 5% lecithin and 50% sucrose) was prepared by mixing 20 or 50 g of liquid lecithin with 450 ml of distilled water for 6 hours to ensure complete dissolution. 500 g of sucrose was added, and the solution was mixed for an additional 1 hour and adjusted to a final volume of 1 L with distilled water.

Liquid Kratom stock was mixed with distilled water (containing 10% or 50% sucrose) or the Premix to yield a final concentration of mitragynine 10 mg/ml (a typical concentration of mitragynine in commercial liquid Kratom shots). Maskers and their concentration are listed in Table B1.

Taste testing was conducted as follows: Two trained panelist were asked to ingest 5-10 ml of the solution and swish it in the oral cavity for 5 seconds ensuring that all surfaces including oral vestibule, gums, roof of the mouth, back of the tongue, and the sublingual space were exposed to the solution. The panelists then expectorated the sample and waited for 5 seconds before thoroughly rinsing the mouth with water. Panelists were asked to cat an unsalted cracker and wait for at least 5 minutes before taste testing the next sample. Each panelist taste tested a maximum of 10 samples per day to prevent desensitization, avoid irritation, and minimize the exposure to active ingredients.

In the first series of experiments five maskers were initially tested in the samples where liquid Kratom stock was mixed with 10% sucrose solution. It was determined that the samples were too bitter and irritating to continue the screen. On the next day new samples were prepared where liquid Kratom stock was mixed with 50% sucrose solution and an additional five maskers were evaluated. Maximizing the sucrose to 50% reduced the irritation and bitterness somewhat but still quickly fatigued the panelists.

On the next day new samples were prepared where liquid Kratom stock was mixed with 50% sucrose solution containing 2, 5, or 10%, w/v of liquid lecithin. Taste testing determined that 2% lecithin had a minor effect on the bitterness, whereas 10% solution was too viscous and carried additional off-flavors. The 5% lecithin solution significantly reduced bitterness and irritation and was selected as the base to conduct the maskers screen. In the next series of experiments the solution containing 5% liquid lecithin, 50% sucrose, and 10 mg/ml mitragynine was prepared and about 40 maskers were evaluated (only 24 listed in Table B1). Among them, three maskers provided significant and robust improvement using the extract and under the conditions of taste test.

TABLE B1
Masking agents for Kratom.

| | Manufacturer's | % | |
|---|---|---|---|
| Product code | description | used | Taste test result |
| MET 4728 | mouthfeel enhancer | 0.8 | minor improvement |
| MET 6985 | masker and sweetness | 2 | significant |
| | enhancer | | improvement |
| MET 9548 | masker | 0.7 | minor improvement |
| MET 9817 | bitter and grassy masker | 0.3 | minor improvement |
| MET 10516 | lingering bitter masker | 0.2 | minor improvement |
| MET 15811 | sweetness enhancer | 0.4 | minor improvement |
| MET 17889 | bitter masker | 0.3 | minor improvement |
| MET 17201 | masker and mouthfeel | 0.4 | no improvement |
| | enhancer | | |
| OSF 1154C | masker | 0.3 | no improvement |
| OSF 1307B | masker | 0.3 | minor improvement |
| OSF 2069C | bitter masker | 0.2 | minor improvement |
| OSF 2070C | caramel flavor, | 0.2 | significant |
| | sweetness enhancer | | improvement |
| OSF 2307C | herbal masker | 0.8 | minor improvement |
| OSF 3006B | masker | 0.4 | minor improvement |
| OSF 4027B | bitter masker | 0.4 | minor improvement |
| OSF 4462 | masker | 0.2 | minor improvement |
| OSF 5475B PDR | fish oil masker | 0.4 | minor improvement |
| OSF 5596 | masker | 0.5 | no improvement |
| OSF 6754B PDR | omega-3 masker | 0.4 | minor improvement |
| OSF 7023A | masker | 0.4 | no improvement |
| OSF 7186B | masker, water sol | 0.4 | significant |
| | version of 7187 | | improvement |
| OSF 7187B | masker | 0.4 | significant |
| | | | improvement |
| OSF 7528A PDR | fatty acid masker | 0.4 | no improvement |
| PRO Y0352A | vanilla | 0.5 | minor improvement |

MET—Foodarom, OSF—OSF Flavors, PRO—Provon.

In the next series of experiments the three maskers OSF 7186B, OSF 2070C, and MET 6985 were tested in different combinations and at various ratios, with one ratio used at 0.5%, 0.4%, and 2%, respectively presented in Table B2.

TABLE B2
Combinations of masking agents for Kratom.

| masker 1 | masker 2 | masker 3 | Rating |
|---|---|---|---|
| MET 6985 | OSF 7186B | OSF 2070C | 1 |
| MET 6985 | OSF 7186B | | 4 |
| OSF 7186B | OSF 2070C | | 2 |
| MET 6985 | OSF 2070C | | 3 |

In the next series of experiments the following Premix was prepared:
MET 6985—2%,
OSF 7186B—0.5%,
OSF 2070C—0.4%,
liquid lecithin—5% w/v,
sucrose—50% w/v
Water, q.s. 1 L.

The Premix was combined with liquid Kratom stock to yield 10 mg/ml final concentration of mitragynine. The premix/Kratom solution was mixed with various flavors to identify those delivering the best match with the masked base and cleanest organoleptic profile. Two trained panelists evaluated the samples. Panelists were asked to pay specific attention to off-flavors that may be enhanced by the added flavor and report their observations as Unremarkable, Match, or No Match and, where obvious, provide a brief description of off-flavors and a note on improvement. The flavor, concentrations, and the results of this screen are provided in Table B3.

TABLE B3
Flavors for Kratom.

| | | % | |
|---|---|---|---|
| Flavor | Product code | used | Description |
| Tiramisu | MET 3223 | 1.0 | Match, must reduce |
| | | | sour notes |
| orange | MET 11579 | 0.5 | Unremarkable |
| mango | MET 1751 | 0.5 | Unremarkable |
| matcha | MET 12044 | 0.3 | Match, need to reduce |
| | | | green notes |
| cake | MET 481 | 0.3 | Unremarkable |
| chocolate caramel | MET 10447 | 0.5 | Unremarkable |
| affogato cheesecake | MET 17270 | 1.5 | Match |
| irish cream | MET 1418 | 1.0 | Unremarkable |
| yogurt | MET 8615 | 1.0 | No match |
| chocolate | MET 1192 | 0.5 | Unremarkable |
| strawberry | MET 3559 | 1.5 | No match |
| black currant | MET 7163 | 1.0 | Match |
| peach/mango | OSF 7896A | 1.2 | Match, peach dominates |
| tropical | MET 11786 | 1.2 | Unremarkable |
| bubble gum | OSF 7297 | 1.2 | Match, slightly bitter |
| | | | aftertaste |
| apricot | OSF 1789C | 1.0 | Match |
| cherry | OSF 1188C | 1.0 | Unremarkable |
| citrus | OSF 3733 | 1.2 | Match |
| apple | MET 2643 | 1.0 | Match, may benefit |
| | | | from cinnamon |
| pina colada | MET 3099 | 1.0 | No match |
| curuba | MET 9667 | 0.8 | Match |
| dulce de leche | MET 8127 | 1.0 | Match, needs more |
| | | | sweetness |
| blue raspberry | MET 00319 | 1.0 | Unremarkable |
| coffee | MET 1463 | 0.5 | Match, may benefit |
| | | | from hazelnut |
| mixed berries | MET 17356 | 0.8 | Match |

MET—Foodarom,
OSF—OSF Flavors

In the next series of experiments two flavors, Passion fruit (Curuba) MET 9667 and Coffee (cappuccino type) MET 1463, were evaluated for further improvement and enhancement of their organoleptic profiles by addition of other flavors, flavor enhancers, and mouthfeel enhancers. About twenty different combinations and ratios were screened for each and the final recipes for the two flavors were identified as presented below. In the case of coffee, addition of Irish cream flavor, as the source of cream and hazelnut notes, and addition of mouthfeel enhancer was beneficial. In the case of curuba, the addition of pineapple type flavor enhancer was beneficial.

TABLE B4
Passion fruit (Curuba) flavored Kratom.

| Flavor function | Product code | Concentration, % |
|---|---|---|
| masker | OSF 7186B | 0.5 |
| masker | MET 6985 | 2 |
| Curuba | MET 9667 | 1 |
| flavor enhancer | OSF 5876 | 0.5 |
| Caramel flavor and sweetness enhancer | OSF 2070C | 0.4 |

TABLE B5
Coffee flavored Kratom.

| Flavor function | Product code | Concentration, % |
|---|---|---|
| masker | OSF 7186B | 0.5 |
| masker | MET 6985 | 2 |
| Cappuccino | MET 1463 | 0.5 |
| Irish cream | MET 460 | 0.4 |
| mouthfeel enhancer | MET 4728 | 0.3 |
| Caramel flavor and sweetness enhancer | OSF 2070C | 0.4 |

### Example C

This experiment demonstrates the concentration and order of addition of Polysorbate 80.

Liquid Kratom stock solutions were prepared as follows: Dry powder extract with mitragynine content 34% (Plant Specimen Supply, LLC, 3161 Major St., Fort Worth, TX 76112) was dispersed in EGC at a final concentration of mitragynine of 67 mg/ml and heated to 70° C. to facilitate dissolution.

Premix was prepared according to the following formula:
Liquid lecithin—5% w/v
Masking agent OSF 7186B—0.5% w/v
Masking agent OSF 2070C—0.4% w/v
Masking agent MET 6985—2% w/v
Sucrose—50% w/v
Water—q.s. 900 ml

Polysorbate 80, to achieve final concentrations shown in Table C1, was added to the liquid Kratom extract, to the Premix, or to the final mix immediately after the extract and the Premix were mixed. Solutions were incubated at room temperature for several weeks and periodically monitored for any signs of phase separation by visual observation. Results are shown in Table C1. In the samples with 0% Polysorbate 80, separation was developed within a few hours. In other samples separation developed in 1-3 days. And samples where Polysorbate 80 was added directly to the extract required the lowest final concentration of Polysorbate 80, possibly indicating that shielding hydrophobic compounds in botanical extracts led to subsequent stability of the emulsion.

TABLE C1
Polysorbate 80 concentration and order of addition.

| Final concentration | Polysorbate | Polysorbate | Polysorbate |
|---|---|---|---|
| of Polysorbate | 80 added to | 80 added to | 80 added to |
| 80, % w/v | Final Mix | premix | liquid extract |
| 0 | separated | separated | separated |
| 0.3 | separated | separated | separated |
| 0.5 | separated | separated | stable |
| 1 | separated | stable | stable |
| 1.5 | stable | stable | stable |

Acceptable Daily Intake (ADI) of Polysorbate 80 is 10 mg/kg body weight per day. These numbers, if applied to a small (60 kg or 132 lb) or large (100 kg or 220 lb) person, translate into 600 and 1000 mg respectively. World Health Organization (WHO) sets the safe daily limit at 25 mg/kg body weight per day.

The formulations presented in the present teaching use Polysorbate 80 at a final concentration of 0.5%. Therefore, liquid shots of 15 ml or 30 ml will deliver 75 mg or 150 mg of Polysorbate 80, respectively. Daily intake of Polysorbate 80 from food in western countries is estimated to be at 12-111 mg/person/day. Therefore, consumption of 1-2 shots of the formulations presented here per day will not exceed ADI.

### Example D

This experiment demonstrates that there is a tremendous variety of bitter maskers and taste maskers offered by flavor houses but most of them are not efficient in reduction of off-flavors in Hemp/Cannabis products. A vast and meticulous screen of maskers had to be conducted to identify a few that combined with each other and with lecithin to deliver a formulation with significantly improved palatability. The example teaches the method for selection of taste maskers, flavors, and their combinations and provides several specific examples of Ready-To-Drink formulations.

Full spectrum hemp extract, as an aqueous solution solubilized with Polysorbate 80 and containing 20 mg/ml CBD, was procured from Nano Cove Industries, 425 S Valley View Blvd Suite 1000, Las Vegas, NV 89118. The solution was diluted 20-fold with distilled water to a final concentration of 1 mg/ml CBD for use in the experiments.

Evaluation of organoleptic properties and taste testing was conducted as follows: Two trained panelists were asked to ingest 5-10 ml of the solution and swish it in the mouth for 10 seconds, ensuring full contact with the back of the tongue, expectorate the sample, and wait for 20 seconds before rinsing the mouth with water. Panelists were asked to eat an unsalted cracker and wait for at least 10 minutes before taste testing the next sample.

In the first experiment hemp extract was mixed with liquid lecithin (5% final concentration) or with water to achieve same dilution. Both solutions were sweetened with sucrose at 7% w/v final concentration. Off-flavors typically attributed to hemp and cannabis products (astringent, hempy, grassy, bitter, irritating) were confirmed and used to assess efficiency of the lecithin and taste maskers. Special attention was paid to the irritation at the back of the tongue, soft palate, and epiglottis. This irritation may or may not be related to astringency. Sometimes hemp/cannabis drinkers describe this as a spasm or a “bite” most strongly felt a few seconds after swallowing the cannabis drink. The presence of lecithin appreciably reduced all the off-flavors and the bite. Hemp extracts in general, as the one used in the current Example, have fewer off-flavors and these off-flavors are significantly milder than off-flavors of Kratom shots. Therefore, most of the subsequent screens were conducted in the absence of lecithin and at relatively low sweetness (7% sucrose).

In the second series of experiments about 28 different taste maskers (only 18 are shown in Table D1) were screened for their ability to reduce off-flavors, in the absence of lecithin, using a No improvement, Minor improvement, or Significant improvement rating system. The results of this screen are presented in Table D1.

TABLE D1
Masking agents for hemp.

| | Manufacturer's | % | |
|---|---|---|---|
| Product code | description | used | Taste test result |
| MET 9548 | bitter masker | 0.3 | no improvement |
| MET 14783 | bitter masker | 0.4 | minor improvement |
| MET 14783 | bitter masker | 0.7 | significant |
| | | | improvement |
| OSF 1307B | bitter masker | 0.37 | no improvement |
| OSF 2069C | bitter masker | 0.37 | minor improvement |
| OSF 2070C | sweetness enhancer | 0.37 | significant |
| | and caramel flavor | | improvement |
| OSF 3006B | bitter masker | 0.5 | minor improvement |
| OSF 4027B | bitter masker | 0.4 | minor improvement |
| OSF 5475B PDR | fish oil masker | 0.4 | minor improvement |
| OSF 5596 | Masker | 0.5 | no improvement |
| TN 101001 | bitter masker | 1 | no improvement |
| TN 201001 | bitter masker | 1 | no improvement |
| TN 301001 | bitter masker | 1 | no improvement |
| OSF 6754B PDR | omega-3 masker | 0.4 | significant |
| | | | improvement |
| OSF 7187B | hemp masker | 0.5 | significant |
| | | | improvement |
| OSF 7186B | hemp masker, water | 0.5 | significant |
| | sol version of 7187 | | improvement |
| OSF 7528A PDR | masker | 0.4 | no improvement |
| OSF 9508A | caramel | | significant |
| | | | improvement |

MET—Foodarom,
OSF—OSF Flavors,
TN—TastesNatural

In the third series of experiments, 22 combinations were prepared of best performing candidate maskers identified above, again with 7% sucrose and no lecithin, and rated for hempy/grassy notes, bitterness, and the bite on a 0-10 scale (0—undetectable, 10—as in the original solution without maskers). The scores for each rated parameter were summed up to identify combinations with the lowest scores. The results are presented in Table D2. Maskers were used at the following final concentrations: OSF 6754B PDR—0.5%, OSF 7186B—0.5%, OSF 2070C—0.4%, OSF 4027B—0.5%, OSF 2069—0.4%, MET 14783—0.8%, OSF 1307B—0.4%, OSF 3006B—0.5%.

TABLE D2
Combinations of masking agents for hemp.

| masker 1 | masker 2 | masker 3 | Hempy | Bitter | Bite | Score |
|---|---|---|---|---|---|---|
| OSF 6754B PDR | OSF 7186B | | 4.5 | 2.5 | 2.5 | 9.5 |
| OSF 6754B PDR | OSF 7186B | OSF 9508A | 5 | 1 | 1 | 7 |
| OSF 6754B PDR | OSF 7186B | OSF 4027B | 6 | 2 | 2 | 10 |
| OSF 6754B PDR | OSF 2069C | | 5 | 3 | 4 | 12 |
| OSF 6754B PDR | OSF 2069C | OSF 7186B | 4.5 | 3 | 2 | 9.5 |
| OSF 6754B PDR | OSF 9508A | | 4.5 | 2 | 4 | 10.5 |
| OSF 6754B PDR | OSF 9508A | OSF 2069C | 6 | 2 | 4 | 12 |
| OSF 6754B PDR | OSF 4027B | | 6 | 4.5 | 8 | 18.5 |
| OSF 6754B PDR | OSF 4027B | OSF 9508A | 5 | 4 | 6 | 15 |
| OSF 6754B PDR | OSF 4027B | OSF 2069C | 7 | 2 | 8 | 17 |
| MET 14783 | OSF 1307B | | 6 | 2 | 4 | 12 |
| MET 14783 | OSF 1307B | OSF 4027B | 5 | 3 | 3 | 11 |
| MET 14783 | OSF 1307B | OSF 7186B | 6 | 3 | 5 | 14 |
| MET 14783 | OSF 1307B | OSF 2069C | 5 | 2 | 3 | 10 |
| MET 14783 | OSF 1307B | OSF 9508A | 5 | 3 | 1 | 9 |
| MET 14783 | OSF 2070C | | 6 | 3 | 3.5 | 12.5 |
| MET 14783 | OSF 3006B | | 6 | 2 | 5 | 13 |
| MET 14783 | OSF 3006B | OSF 1307B | 4 | 2 | 2 | 8 |
| MET 14783 | OSF 3006B | OSF 4027B | 4 | 1 | 2.5 | 7.5 |
| MET 14783 | OSF 3006B | OSF 7186B | 5 | 1 | 2.5 | 8.5 |
| MET 14783 | OSF 3006B | OSF 2069C | 7 | 2 | 6 | 15 |
| MET 14783 | OSF 3006B | OSF 9508A | 6 | 1 | 8 | 15 |

In the fourth series of experiments, the eight lowest score combinations of the maskers from Table D2 were rated against each other for the lowest bite and overall palatability using a 5 point rating system (1—best, 5—worst). The results are presented in Table D3.

TABLE D3
Combinations of masking agents for hemp.

| masker 1 | masker 2 | masker 3 | Rating |
|---|---|---|---|
| OSF 6754B PDR | OSF 7186B | | 4 |
| OSF 6754B PDR | OSF 7186B | OSF 9508A | 1 |
| OSF 6754B PDR | OSF 7186B | OSF 4027B | 3 |
| OSF 6754B PDR | OSF 2069C | OSF 7186B | 4 |
| MET 14783 | OSF 1307B | OSF 9508A | 4 |
| MET 14783 | OSF 3006B | OSF 1307B | 2 |
| MET 14783 | OSF 3006B | OSF 4027B | 5 |
| MET 14783 | OSF 3006B | OSF 7186B | 3 |

In the fifth experiment four combinations from Table D3 were selected for their performance and diversity and tested in the absence and presence of 5% w/v liquid lecithin. 7% w/v sucrose was used in all samples. In each case addition of lecithin, as in the first experiment, further reduced the off-flavors and significantly improved the overall palatability. The lecithin containing samples were rated against each other (1—best, 4—worst) are provided in Table D4.

TABLE D4
Combinations of masking agents for hemp.

| masker 1 | masker 2 | masker 3 | Rating |
|---|---|---|---|
| OSF 6754B PDR | OSF 7186B | OSF 9508A | 1 |
| OSF 6754B PDR | OSF 7186B | OSF 4027B | 2 |
| MET 14783 | OSF 3006B | OSF 1307B | 4 |
| MET 14783 | OSF 3006B | OSF 7186B | 3 |

In the sixth, final series of experiments, one combination of maskers (OSF 6754B PDR—0.5%, OSF 7186B—0.5%, OSF 2070C—0.4%) with 5% w/v liquid lecithin and 9% sucrose was prepared. The solution was mixed with various flavors to identify those delivering the best match with the masked base and cleanest organoleptic profile.

Two trained panelists evaluated the samples as above except that time between taste testing the samples was reduced to two minutes. Panelists were asked to pay specific attention to off-flavors that may be enhanced by the added flavor and report their observations as Unremarkable, Match, or No Match and provide a brief description of off-flavors and a note on improvement. The results of this screen are provided in Table D5.

TABLE D5
Flavors for hemp.

| Flavor | Product code | % used | Description |
|---|---|---|---|
| guava | MET 1379 | 1 | No match, good start but hempy |
| | | | aftertaste |
| rum raisin | MET 13232 | 0.6 | No match, iodine taste |
| mojito | MET 1939 | 0.6 | Match |
| coconut | MET 239 | 0.4 | Unremarkable |
| mango | MET 6368 | 0.6 | No match, brings out hemp |
| irish cream | MET 460 | 0.4 | Match, needs more sweetness |
| irish cream | MET 460 | 0.8 | No match, brings out green |
| guanabana | MET 11068 | 0.7 | Match, may benefit from more |
| | | | flavor |
| guanabana | MET 11068 | 1.2 | Match, may benefit from more |
| | | | sweetness |
| jack fruit | MET 5213 | 0.6 | No match, brings out hemp |
| fig | MET 4730 | 0.8 | Match |
| pina colada | MET 3099 | 0.4 | No match, brings out green |
| chocolate | MET 1947 | 0.6 | No match, brings out green |
| banana | MET 177 | 0.6 | Match, may benefit from more |
| | | | sweetness |
| raspberry | MET 1336 | 0.4 | Match, may benefit from more |
| | | | flavor and sweetness |
| black currant | MET 7163 | 0.6 | Unremarkable |
| dulce de leche | MET 8127 | 1.4 | Match |

### Example E

This experiment demonstrates that the taste masking method and formulation works on other botanical extracts besides Kratom and hemp.

An extract from Water Hyssop Bacopa monnieri (Bacognize® from Verdure Sciences, 17150 Metro Park Court, Noblesville, IN, 46060, USA). The extract is a dark brown powder with acute bitterness, sour/acidic off-flavor, irritation, and characteristic burnt leaf smell and taste. The phytochemicals in Bacognize® demonstrated activity on serotonin (5HT-1a) receptor and chemically are triterpenoid saponins known as bacosides.

An extract from Tongkat Ali or Longjack Euricoma longifolia (LJ100®, from HP Ingredients, 707 24^{th} Ave. W. Bradenton, Florida 34205 USA). The extract is a dark brown powder with acute bitterness, sour/acidic off-flavor, irritation, and acrid herbal smell and taste. LJ100® E. longifolia to be marketed, must be standardized to eurycomanone content between 0.8%-1.5%, polysaccharides above 30%, protein above 20% and glycosaponin above 40%.

Since the main active ingredients (phytochemicals) in Bacognize® and LJ100® are not alkaloids as in Kratom extract and purified from non-related plant species, the extraction procedure and overall composition of tastants is different enough from Kratom and hemp, to provide a meaningful assessment of the present teaching's applicability to a broader selection of botanicals.

Liquid Bacognize® stock solution was prepared as follows: 3.33 g of Bacognize® dry powder extract was dispersed in 16 ml of EGP solvent, heated to 70° C. and vortexed to facilitate dissolution. Polysorbate 80 was added to a final concentration of 5.5% and the volume was adjusted to 20 ml with EGC to achieve a final concentration of the extract 167 mg/ml. Liquid LJ100® stock solution was prepared following the same procedure.

The recommended dose supported by several clinical studies is 300-600 mg of Bacognize® powder extract per day, usually split in two doses (Lopresti A, Smith S, Ali S, Metse A, Kalns J, Drummond P. Effects of a Bacopa monnieri extract (Bacognize®) on stress, fatigue, quality of life and sleep in adults with self-reported poor sleep: A randomized, double-blind, placebo-controlled study. J Functional Foods, 2021 Oct; 85:104671) (Calabrese C, Gregory W L, Leo M, Kraemer D, Bone K, Oken B. Effects of a standardized Bacopa monnieri extract on cognitive performance, anxiety, and depression in the elderly: a randomized, double-blind, placebo-controlled trial. J Altern Complement Med. 2008 July; 14 (6): 707-13), therefore 1-4 ml of such liquid Bacognize® stock would have to be consumed in a final formulation.

The manufacturer of LJ100® recommended 200 mg as the maximum required daily dose.

A total of 8 samples were prepared. 1.5 ml of liquid Bacognize® or LJ100® stock solutions (an equivalent of 250 mg of dry extract) were mixed with 8.5 ml of Premix (zero sugar, 6% lecithin, see composition in Example K) or with 8.5 ml of distilled water, to yield a final concentration of 25 mg/ml, or with 28.5 ml Premix or water to yield a final concentration of 8.3 mg/ml.

Samples Bacognize® or LJ100@ were subjected to organoleptic evaluation by two trained panelists and rated against each other within the extract group for bitterness, irritation, astringency, sour/acidic off-flavor and overall acceptance using a 1-10 scale with 10 being strong and 0 undetectable. Taste test results are presented in Table E1.

RealTurkey Tail™, RealShiitake™, RealCordiceps™, RealReishi™, RealLionsMane™, RealChaga™ (all at 8:1 extract ratio), and Maitake mushroom (10:1 ratio) extracts were procured from Nammex, Box 1780, Gibsons, BC Canada, VON 1V0. All extracts were blended, in equal ratios. Two samples of 750 mg of the blend were directly mixed with 15 ml of premix or water for reference. The sample dissolved in water appeared as a dark brown colored suspension that, after sitting on the bench, became a densely colored solution with significant amount of sediment on the bottom, indicating partial dissolution. The sample dissolved in the Premix also became heavily colored and developed the same amount of sediment. The dosage of botanical extract, at an 8:1 extract ratio, is equal to about 6 g of dried mushrooms that can be delivered in one shot. This is above a typical commercial drink of ˜2 g of dried mushrooms per serving. Mushroom drinks are typically sold as heavily spiced teas that still taste like mushrooms.

Mushroom samples were shaken to ensure homogeneity and analyzed for organoleptic properties by two trained panelists as described in other Examples. The sample dissolved in water was mildly bitter but carried strong brothy and mushroom notes that made the drink aversive. Taste test results are presented in Table E1. Mushroom notes in the sample with Premix were barely detectable and the drink was very acceptable.

TABLE E1
Masking of botanical extracts by Premix.

| | Dilution | Early bitterness/ | | | Sour/ | Overall |
|---|---|---|---|---|---|---|
| | media | Bitter aftertaste | Irritant | Astringent | acidic | acceptance |
| Bacognize ® 25 mg/ml | Water | 10/7 | 10 | 8 | 10 | No |
| Bacognize ® 25 mg/ml | Premix | 3/2 | 4 | 3 | 4 | No |
| Bacognize ® 8 mg/ml | Water | 8/5 | 8 | 4 | 8 | No |
| Bacognize ® 8 mg/ml | Premix | 0/0 | 0 | 1 | 2 | Acceptable |
| LJ100 ® 25 mg/ml | Water | 10/10 | 10 | 10 | 10 | No |
| LJ100 ® 25 mg/ml | Premix | 8/6 | 6 | 8 | 7 | No |
| LJ100 ® 8 mg/ml | Water | 9/9 | 9 | 9 | 9 | No |
| LJ100 ® 8 mg/ml | Premix | 4/5 | 3 | 4 | 3 | No |
| Mushroom extract | Water | 3/1 | 3 | 4 | 2 | No |
| Mushroom extract | Premix | 0/0 | 0 | 0 | 0 | Acceptable |

The taste masking system used was optimized for Kratom, not mushrooms, Bacognize®, or LJ 100®; nevertheless, a strong inhibition of aversive tastes was achieved for all three extracts. Mushrooms and Bacognize® were masked very efficiently and yielded organoleptically acceptable products without any changes to the formula at concentrations high enough to deliver the extract in a 15-30 ml liquid shot. Masking of LJ 100® was less efficient but very significant. Due to the lower dose required the concentration of the extract in the drink can be further reduced at least two-fold, thus improving the taste, and maskers can be optimized to yield a palatable liquid shot of LJ 100®. This experiment demonstrates that the method of the present teaching is widely applicable for taste masking of concentrated unpalatable botanicals.

### Example F

This Example demonstrates that the taste masking method and formulation work on other bitter tastants, such as caffeine and antibiotics.

Caffeine is frequently used as an example of a bitter compound in drinks and it appears to act in bitter taste receptor-independent pathways. It stimulates “taste” receptors in non-gustatory cells (Poole R L, Tordoff M G. The Taste of Caffeine. J Caffeine Res. 2017 Jun. 1; 7 (2): 39-52). It is therefore a non-prototypical bitterant that can further demonstrate the breadth of the method presented here.

The low concentration threshold for tasting the caffeine bitterness in an aqueous solution is ˜0.117 mg/mL (Liszt K I, Ley J P, Lieder B, Behrens M, Stöger V, Reiner A, Hochkogler C M, Köck E, Marchiori A, Hans J, Widder S, Krammer G, Sanger G J, Somoza M M, Meyerhof W, Somoza V. Caffeine induces gastric acid secretion via bitter taste signaling in gastric parietal cells. Proc Natl Acad Sci USA. 2017 Jul. 25; 114 (30): E6260-E6269).

Caffeine was purchased from Bulk Supplements (7511 Eastgate Rd, Henderson, NV 89011). A 75 mg/ml aqueous solution was diluted in either water or Premix (zero sugar, 6% lecithin, see composition in EXAMPLE K) to yield the concentrations shown in Table F1. The samples were rated for bitterness by two trained panelists.

Caffeine was not detectable in the Premix up until at least 7.5 mg/ml, i.e., 64 times above the threshold of detection in water indicating a powerful caffeine masking effect of the method.

TABLE F1
Masking of caffeine by Premix.

| | Dilution media | Bitterness |
|---|---|---|
| Caffeine 3.75 mg/ml | Water | 7 |
| Caffeine 3.75 mg/ml | Premix | 0 |
| Caffeine 7.5 mg/ml | Water | 8 |
| Caffeine 7.5 mg/ml | Premix | 0 |
| Caffeine 15 mg/ml | Water | 10 |
| Caffeine 15 mg/ml | Premix | 2 |

Azithromycin is a macrolide class antibiotic with a typical daily dose of 500 mg. It is known to be bitter and several attempts to create its palatable formulation were reported, including micelles (Huang R, Zhang Y, Wang T, Shen L, Zhang Z, Wang Y, Quan D. Creation of an assessment system for measuring the bitterness of azithromycin-containing reverse micelles. Asian J Pharm Sci. 2018 July; 13 (4): 343-352).

Ciprofloxacin is a fluoroquinolone class antibiotic with a typical daily dose of 500 mg. It is known to be acutely bitter and several attempts to create its palatable formulation were reported (Pisal S, Zainnuddin R, Nalawade P, Mahadik K, Kadam S. Molecular properties of ciprofloxacin-Indion 234 complexes. AAPS PharmSciTech. 2004 Sep. 22; 5 (4): e62).

Azithromycin dihydrate and Ciprofloxacin base were samples from Zhejiang Guobang Pharmaceutical Co Ltd (No. 6 Weiwu Road, Hangzhou Gulf Shangyu Economic and Technological development zone, Zhejiang, China).

Antibiotic powders were suspended in either water or the premix (zero sugar, 6% lecithin, see composition in EXAMPLE K) to deliver the 500 mg dose in 100 ml of the premix. Azithromycin in water was mildly aversive due to strong bitterness. The bitterness was completely masked by the premix and the drink was rated as highly palatable. Ciprofloxacin was extremely bitter and highly aversive in water but mildly bitter and mildly aversive in the premix. Adding Chocolate flavor MET 1947 to the premix at final concentration of 0.8% made the antibiotic drink Acceptable.

Antibiotics are antibacterial chemotherapeutics including emergency medicine where formulated dosage forms may not be available (Sadrich N, Brower J, Yu L, Doub W, Straughn A, Machado S, Pelsor F, Martin E S, Moore T, Reepmeyer J, Toler D, Nguyenpho A, Roberts R, Schuirmann D J, Nasr M, Buhse L. Stability, dose uniformity, and palatability of three counterterrorism drugs-human subject and electronic tongue studies. Pharm Res. 2005 Oct; 22 (10): 1747-56). The present teaching is equally applicable to any orally dosed unpalatable pharmaceutical independently from indication, drug class, or any other classification. Rapid development of a palatable liquid, powder, semi-solid, or solid product incorporating an unpalatable pharmaceutical can be achieved following the present teaching. A drink may be prepared with a liquid or dry premix as described in Example M. Some of the examples of unpalatable active pharmaceutical ingredients that may be formulated according to the present teaching are: analgesics, antacids, anabolics, anxiolytics antiarrhythmic, antibacterials and antibiotics, anticoagulants and thrombolitics, anticonvulsants, antidepressants, antidiarrheals, antiemetics, antifungals, antihistamines, antihypertensives, anti-inflammatoires, antineoplastics, antipsychotics, antipyretics, antivirals, beta-blockers, bronchodilators, cold cures, corticosteroids, cough suppressants, cytotoxics, decongestants, diuretics, expectorants, hormones, hypoglycemics, immunosuppressives, laxatives, muscle relaxants, sedatives, sex hormones, sleeping drugs, tranquilizers, vitamins, etc. A few specific non-limiting examples of unpalatable active pharmaceutical ingredients are alprazolam, amphetamines, buprenorphine, carisoprodol, cyclobenzaprine, codeine, diazepam, diclofenac, lorazepam, methylphenidate, tianeptine, tramadol, etc. As further demonstrated in Example M, the approach is equally applicable to pills, tablets, capsules, and other liquid, powder, semi-solid and solid oral dosage forms, with obvious exceptions such as enterically coated pills and the like.

### Example G

This Example compares the effect of two acidulants, citric vs phosphoric acid, on sour/acidic notes of Kratom in otherwise identical formulations. Both acidulants are commonly used in foods, supplements, and pharmaceuticals, but only citric acid can impart tartness sought in certain products and flavors. Citrate's tartness, in the context of liquid Kratom shot, may be counterproductive and will enhance sour/acidic off-flavors of Kratom.

EGP and EGC solvents were prepared according to the recipes in Table G1.

TABLE G1
Compositions of citrate- and phosphate- containing solvents.

| | | |
|---|---|---|
| Ingredients | EGC | EGP |

| | | | | |
|---|---|---|---|---|
| 95% Ethanol | 190 | ml | 190 | ml |
| water | 260 | ml | 343.6 | ml |
| Glycerol | 450 | ml | 450 | ml |
| Citric acid, 50% w/v | 100 | ml | | |
| Phosphoric acid, 75% w/v | | | 16.4 | ml |

| | | |
|---|---|---|
| pH | 3.8 | 3.8 |

| | | | | |
|---|---|---|---|---|
| final volume | 1000 | ml | 1000 | ml |

Liquid Kratom stock solutions were prepared as follows: Dry powder extract with mitragynine content 70% (from Pure Kratom, LLC) were dispersed in EGC or EGP at final concentrations of mitragynine of 80 mg/ml and heated to 70° C. to facilitate dissolution. Polysorbate 80 was added to a final concentration of 5.5%.

Premix, Transparent 53 formula with 4% de-oiled lecithin was prepared as below and mixed for 6 hours to ensure complete dissolution of the lecithin.
De-oiled lecithin—4% w/v
Glycerol—10% v/v
Propylene glycol—7.5% v/v
Ethanol—12.5% v/v
Masking agent OSF 7186B—0.5% w/v
Masking agent OSF 2070C—0.4% w/v
Masking agent MET 6985—2% w/v
Allulose—56% w/v
Sodium saccharin—0.025% w/v
Monk fruit extract, 25% mogroside V—0.15% w/v
Water—q.s. 1000 ml

87 ml of the Premix and 13 ml of liquid Kratom stocks were mixed to achieve two 100 ml samples (with EGC-citrate vs EGP-phosphate) at a final concentration of mitragynine 10.4 mg/ml.

20 ml aliquots of each sample were retained (pH 4.1), 80 ml were titrated with 10 N KOH solution to pH 6.0. All four samples were rated against each other by two trained panelists for overall palatability and presence of sour/acidic notes and tingling. The results of the taste test are presented in Table G2.

TABLE G2
Palatability vs acidulant.

| | Palatability. | Sour/acidic | |
|---|---|---|---|
| | 4-best, 1-worst | off-flavor | Tingling |
| EGP - phosphate, pH 4.1 | 3 | mild | mild |
| EGC - citrate, pH 4.1 | 1 | strongest | strongest |
| EGP - phosphate, pH 6.0 | 4 | minimal | minimal |
| EGC - citrate, pH 6.0 | 2 | strong | strong |

### Example H

This Example screens various candidate formulations with the purpose of developing transparent and stable formula with good taste masking properties and minimum or no off-flavors imparted by the solvent system used. Only a part of all combinations screened is shown.

10 ml of premix of each formulation was prepared in a 15 ml plastic conical tube according to the recipes in Table H1. Allulose was used as 80% w/v solution in water. Sorbitol was used as 70% w/v solution on water. Samples were briefly vortexed, heated on a 70° C. water bath and vortexed with periodic reheating until complete dissolution of de-oiled lecithin (3-5 minutes). Samples of premix were allowed to cool to room temperature. 1 ml of liquid Kratom stock solution (100 mg/ml) was mixed with 9 ml premix samples to achieve a final concentration of mitragynine 10 mg/ml.

Liquid Kratom stock solution: Dry powder extract with mitragynine content 70% (from Pure Kratom, LLC) was dissolved in an EGP solvent at final concentration of mitragynine at 100 mg/ml. Heated to 70° C. to facilitate dissolution. Polysorbate 80 was added to a final concentration of 5.5%.

Maskers and high intensity sweeteners were added to achieve final concentrations as follows:
Masking agent OSF 7186B—0.5% w/v
Masking agent OSF 2070C—0.4% w/v
Masking agent MET 6985—2% w/v
Sodium saccharin—0.05% w/v
Neohesperidin dihydrochalcone—0.025% w/v
Monk fruit extract, 25% mogroside V—0.15% w/v

Samples were mixed by inversion and vortexing. One ml aliquots were placed in Eppendorf tubes for evaluation by visual observation for transparency, and phase separation upon storage. The remaining 9 ml of each sample, where indicated, were split in two 4.5 ml aliquots and used for organoleptic evaluation by two trained panelists.

Note that premixes themselves, without the liquid kratom stock added, generally demonstrated lower transparency than after addition of the kratom stock. A separate experiment confirmed that addition of Polysorbate 80 and EGP solvent both contributed to improved transparency.

TABLE H1
Development of clear formulation.

| | | | | | | Transparency, | | |
|---|---|---|---|---|---|---|---|---|
| Ethanol | | Sorbitol | Allulose | Glycerol | | 1-transparent, | Phase | |
| 95%, ml | PG, ml | 70%, ml | 80%, ml | 100%, ml | H₂O, ml | 10-haxy | separation | Taste notes |
| | 3 | 7 | | | | 4 | | bitter, too viscous |
| | 3 | | | 7 | | 4 | yes | warming |
| 3 | | | | 7 | | 2 | | alcohol burn |
| 3 | | 7 | | | | 2 | yes | alcohol burn |
| 1.25 | 1 | | | 5.6 | 2.15 | 1 | | sweet, warming |
| 1.25 | | | 5.6 | | 3.15 | 5 | | sweet |
| | 2 | | 5.6 | | 2.4 | 3 | | chemical, soapy |
| | 2 | | | 5.6 | 2.4 | 4 | | sweet, warming |
| 1 | 1 | | 5 | 3 | | 2 | yes | chemical taste |
| 2 | | | 6 | | 2 | 2 | | not done |
| 2 | | | 4 | | 4 | 5 | | not done |
| | | | 10 | | | 5 | yes | not done |
| 0.5 | | | 8 | | 1.5 | 6 | | not done |
| 1 | | | 8 | | 1 | 4 | | not done |
| | | | 10 | | | 3 | yes | not done |
| 0.5 | | | 8.5 | | 1 | 5 | | not done |
| 0.5 | | | 7 | | 2.5 | 6 | | not done |
| | | | 6 | | 4 | 7 | | not done |
| 1 | | | 6 | | 3 | 5 | | not done |
| 1 | 1 | | 7 | 1 | | 1 | | sweet, fruity |
| 1 | 1 | | 6 | 2 | | 1 | | sweet, warming |
| 1 | 1 | | 7 | | 1 | 3 | | not done |
| 2 | | | 6 | 2 | | 1 | | alcohol taste |
| 1.5 | 0.5 | | 8 | | | 1 | | sweet, good |
| 2 | | | 8 | | | 1 | | alcohol taste |
| 1.25 | 0.75 | | 7 | 1 | | 1 | | sweet, good |

PG—propylene glycol

### Example I

This Example demonstrates that taste masking effect is directly proportional to lecithin concentration.

Premixes (5% de-oiled lecithin or no lecithin) were prepared according to the following formula:
De-oiled lecithin—5% w/v
Masking agent OSF 7186B—0.5% w/v
Masking agent OSF 2070C—0.4% w/v
Masking agent MET 6985—2% w/v
Glycerol—10% w/v
Allulose—10% w/v
Sodium saccharin—0.05% w/v
Neohesperidin dihydrochalcone—0.025% w/v
Monk fruit extract, 25% mogroside V—0.15% w/v
Water, q.s. 50 ml

Mixing was performed for 6 hours to allow complete dissolution of lecithin.

Liquid Kratom stock solution: Dry powder extract with mitragynine content 70% (from Pure Kratom, LLC) was dispersed in an EGP solvent at a final concentration of mitragynine of 100 mg/ml. Heated to 70° C. to facilitate dissolution. Polysorbate 80 was added to a final concentration of 5.5%.

10 ml aliquots of samples with various concentrations of lecithin were prepared by mixing 5% and 0% lecithin solutions as shown in Table I1 and 1.1 ml of liquid Kratom stock was added to yield ˜10 mg/ml concentration of mitragynine.

Samples were analyzed for organoleptic properties by two trained panelists as described in other Examples. The panelists were asked to provide overall rating of the samples, provide a brief description, paying specific attention to sour/acidic taste/irritation commonly described as the Kratom taste.

TABLE I1
Lecithin concentration vs palatability.

| Final | 5% | No | | Palatability |
|---|---|---|---|---|
| lecithin | lecithin | lecithin | | rating, |
| concentra- | premix, | premix, | | 10-best, |
| tion, % | ml | ml | Taste testing results | 1-worst. |
| 5 | 10 | 0 | Fruity, sweet, slight | 10 |
| | | | green taste | |
| 4 | 8 | 2 | Minor irritation, sweet, | 8 |
| | | | residual Kratom taste | |
| 3 | 6 | 4 | Irritation, residual | 4 |
| | | | bitterness and Kratom, sour | |
| 2 | 4 | 6 | Major irritation, bitter, | 1 |
| | | | strong Kratom, sour | |
| 1 | 2 | 8 | Major irritation and acute | 1 |
| | | | bitterness, strong Kratom, | |
| | | | sour | |

The experiment demonstrated that the taste masking effect of lecithin is concentration dependent and, in the case of de-oiled lecithin, at least 3% concentration was required for noticeable improvement of organoleptic properties of Kratom extract at 10 mg/ml mitragynine.

### Example J

This Example tests the pH of commercial liquid Kratom shots.

Products were purchased in local smoke shops, online, or received as samples from manufacturers and distributors at trade shows. Some of the products did not have ingredients listed, neither on the bottle or packaging, nor the website.

Unstable (drifting) pH readings, likely due to high content of organic solvents in the formula, are marked as ND. Such samples were diluted 1:1 with distilled water to obtain readings (still unstable in most cases).

TABLE J1
pH of commercial Kratom preparations.

| | Brand, supplier, | | Notes on listed ingredients, physical appearance, |
|---|---|---|---|
| Product Name | distributor, or website | pH | and pH measurement. |
| tusk green apple | tuskkratom.com | 3.7 | low mitragynine concentration formula |
| speed 100+ | speed100plus.com | 3.5 | |
| sugar shots | Pastel cartel, Naturally dist [symbol] | 4.9 | low mitragynine concentration formula |
| MIT45 | mit45.com | 3.3 | |
| 300 ultra | green shores nutra | 4.5 | Crude suspension |
| green leaf | glkusa.com | 5.0 | Crude suspension |
| my bliss | Five Wise LLC | 3.0 | Crude suspension |
| my45 | Five Wise LLC | 4.7 | |
| dave's brew | mittherapy.com | 3.1 | Crude suspension |
| mitra 165 nano | american pride llc | 3.9 | |
| mit45 super K | mit45.com | 3.2 | Crude suspension |
| kr8om | kr8om.com | 3.6 | |
| maeng da | kravekratom.com | 3.2 | |
| magic potion lipht | lipht.com | ND | Suspension with agglomerates, pH reading unstable, |
| | | | diluted in water pH 4.2 |
| crave+ butterscotch | sigdistro.com | ND | pH reading unstable, diluted in water pH 4.4 |
| luxx black nano | kloud distributors (confirm [symbol] | ND | Propylene Glycol based formula, pH reading |
| | | | unstable, diluted in water pH 5.1 |
| ultimate kratom | ultimateherbals.com | ND | pH reading unstable, likely organic solvent, |
| | | | immediate precipitation upon dilution in water, |
| | | | diluted in water pH 10.5 formed clots |
| mit freeze | lifestyle marketing llc | ND | Propylene Glycol based formula, pH reading |
| | | | unstable, immediate precipitation upon dilution in |
| | | | water |
| nano kratom prime | triumph botanicals | ND | Polyethylene Glycol formula, unstable pH reading. |
| | | | Diluted in water pH 6.0 |
| tropix nano | ktropix.com | ND | Polyethylene Glycol formula, unstable pH reading. |
| | | | Diluted in water pH 5.6 |

[symbol]

indicates data missing or illegible when filed

### Example K

This Example determines if lecithins (liquid or de-oiled) can improve solubility of Kratom extracts and mitragynine at higher pH. The samples also enabled evaluation of formulations, lecithins, and pH conditions on liposome particle size, entrapment efficiency, and overall organoleptic properties.

Liquid Kratom stock solution. Dry powder extract with mitragynine content 70% (from Pure Kratom, LLC) was dispersed in EGP solvent at final concentration of mitragynine 80 mg/ml and heated to 70° C. to facilitate dissolution. Polysorbate 80 was added to a final concentration of 5.5% w/v.

Zero sugar with 4% de-oiled lecithin premix (the resulting percentages in the premix are about 10% higher than cited and intended to reflect the final percentages upon dilution with liquid Kratom stock solution). The premix was mixed for 6 hours to ensure complete dissolution of the lecithin.
De-oiled lecithin—4% w/v
Masking agent OSF 7186B—0.5% w/v
Masking agent OSF 2070C—0.4% w/v
Masking agent MET 6985—2% w/v
Glycerol—10% w/v
Allulose—10% w/v
Sodium saccharin—0.025% w/v
Neohesperidin dihydrochalcone—0.025% w/v
Monk fruit extract, 25% mogroside V—0.15% w/v
Water—q.s. 900 ml

Liquid Kratom stock solution was mixed with the premix (Zero sugar, 4% de-oiled lecithin) to achieve a final concentration of mitragynine 10.4 mg/ml.

200 ml of the resulting solution (Zero sugar, 4% de-oiled lecithin, 10.4 mg/ml mitragynine) was placed in a glass beaker on a magnetic stirrer and pH electrode was inserted to allow constant pH monitoring (initial pH was 4.1). To the constantly stirred solution, 10 N KOH was added dropwise, and 20 ml aliquots were drawn at the following pH: 4.1, 5.0, 6.0, 7.0, 8.2. The solution was monitored by visual observation to determine any change in turbidity and color. Rapid increase of turbidity and khaki color intensity was observed at pH 6.5 indicating precipitation of Kratom extract. The aliquots were used for evaluation of organoleptic properties, measurements of Optical Density (300-800 nm), Particle Size, and entrapment efficiency (Dialysis-HPLC). All samples, including those showing precipitation of Kratom extracts (pH 6.5, 7.0, and 8.2) remained stable for at least 3 months, showing no signs of phase separation.

Zero sugar with 6% liquid lecithin premix (the resulting percentages in the premix are about 10% higher than cited and intended to reflect the final percentages upon dilution with liquid Kratom stock solution). The premix was mixed for 6 hours to ensure complete dissolution of the lecithin.
Liquid lecithin—6% w/v
Masking agent OSF 7186B—0.5% w/v
Masking agent OSF 2070C—0.4% w/v
Masking agent MET 6985—2% w/v
Glycerol—10% w/v
Allulose—10% w/v
Sodium saccharin—0.05% w/v
Neohesperidin dihydrochalcone—0.025% w/v
Monk fruit extract, 25% mogroside V—0.15% w/v
Water—q.s. 900 ml

Liquid Kratom stock solution was mixed with the premix (Zero sugar, 6% liquid lecithin) to achieve a final concentration of mitragynine 10.4 mg/ml.

200 ml of the resulting solution (Zero sugar, 6% de-oiled lecithin, 10.4 mg/ml mitragynine) was placed in a glass beaker on a magnetic stirrer and pH electrode was inserted to allow constant pH monitoring (initial pH was 4.3). To the constantly stirred solution, 10 N KOH was added dropwise, and 20 ml aliquots were drawn at the following pH: 4.3, 5.0, 6.0, 7.0, 8.2, 9.0, 10.1. The solution was monitored by visual observation to determine any change in turbidity and color. Visible increase of khaki color intensity was observed at pH 10.0 indicating precipitation of Kratom extract. The aliquots were used for evaluation of organoleptic properties, measurements of Optical Density (300-800 nm), Particle Size, and entrapment efficiency (Dialysis-HPLC). All samples had a thin, froth-like layer floating at the top. This froth-like layer remained white in pH 4.3 and 5.0 samples, whereas at higher pH it was colored as the rest of the sample (light brown, lecithin-like) and included few visible oil drops. The bulk of all samples (liquid part), remained stable for at least 3 months, showing no signs of phase separation. Sample at pH 10.1 developed a small amount of precipitate at the bottom after about 6 weeks. Oil separation in these samples. This is the only set of samples in this Example that had separation of oily phase on top and the only formulation that is fully aqueous and uses liquid lecithin, which is 50% oil. The separation is obvious only at pH 6.0 and above, potentially indicating near zero zeta potential which leads to fusion of some of the liposomes. This was fixed by either blending liquid and de-oiled lecithin, or addition of small amount of cosolvents (ethanol or Polysorbate 80).

To determine pH of Kratom precipitation in the absence of lecithins the Premixes were prepared as above (Zero sugar without lecithins) or below (Transparent 53 without lecithins).

Liquid Kratom stock solutions were mixed with the premixes (Transparent 53, no lecithin or zero sugar, no lecithin) to achieve a final concentration of mitragynine 10.4 mg/ml.

200 ml of the resulting solutions were placed in a glass beaker on a magnetic stirrer and pH electrode was inserted to allow constant pH monitoring (initial pH was 3.7). To the constantly stirred solution, 10 N KOH was added dropwise. The solution was monitored by visual observation to determine any change in turbidity and color. In the Transparent formulation, the first signs of precipitation were observed at pH 6.1. At pH 6.5 the solution became opaque with intense khaki color indicating complete precipitation of Kratom extract. In the Zero sugar formulation the first signs of precipitation were observed at pH 5.6. At pH 6.0 the solution became opaque with intense khaki color indicating complete precipitation of Kratom extract.

Transparent 53 with 4% de-oiled lecithin premix. The premix was mixed for 6 hours to ensure complete dissolution of the lecithin.
De-oiled lecithin—4% w/v
Glycerol—10% v/v
Propylene glycol—7.5% v/v
Ethanol—12.5% v/v
Masking agent OSF 7186B—0.5% w/v
Masking agent OSF 2070C—0.4% w/v
Masking agent MET 6985—2% w/v
Allulose—56% w/v
Sodium saccharin—0.025% w/v
Monk fruit extract, 25% mogroside V—0.15% w/v
Water—q.s. 1000 ml

Liquid Kratom stock solution was mixed with the premix (Transparent 53, 4% de-oiled lecithin) to achieve final concentration of mitragynine 10.4 mg/ml. After mixing with the liquid Kratom stock solution the resulting percentages in the premix were about 10% lower than cited in the premix recipe above.

200 ml of the resulting solution (Transparent 53, 4% de-oiled lecithin, 10.4 mg/ml mitragynine) was placed in a glass beaker on a magnetic stirrer and pH electrode was inserted to allow constant pH monitoring (initial pH was 4.1). To the constantly stirred solution, 10 N KOH was added dropwise, and 20 ml aliquots were drawn at the following pH: 4.1, 5.0, 6.0, 7.0, 8.0. The solution was monitored by visual observation to determine any change in turbidity and color. At pH 4.1, 5.0, 6.0 the solution was transparent. At pH 7.0 the solution developed some turbidity and became translucent. At pH 8.0 the solution was opaque indicating complete precipitation of Kratom extract. The aliquots were used for evaluation of organoleptic properties, measurements of Optical Density (300-800 nm), Particle Size, and entrapment efficiency (Dialysis-HPLC). Both solutions that developed turbidity and color (pH 7.0 and 8.0) in about one week developed a form of easily breakable aggregates settling on the walls of the tube and at the bottom. After about 6 weeks the aggregates turned into solid formations that similar to amorphous crystals growing on the tubes' walls.

Transparent 53 with 6% liquid lecithin premix. The premix was mixed for 6 hours to ensure complete dissolution of the lecithin. The resulting Premix was not transparent but translucent due to use of liquid lecithin.
Liquid lecithin—6% w/v
Glycerol—10% v/v
Propylene glycol—7.5% v/v
Ethanol—12.5% v/v
Masking agent OSF 7186B—0.5% w/v
Masking agent OSF 2070C—0.4% w/v
Masking agent MET 6985—2% w/v
Allulose—56% w/v
Sodium saccharin—0.05% w/v
Monk fruit extract, 25% mogroside V—0.15% w/v
Water—q.s. 1000 ml

Liquid Kratom stock solution was mixed with the premix (Transparent 53, 6% liquid lecithin) to achieve final concentration of mitragynine 10.4 mg/ml. After mixing with the liquid Kratom stock solution the resulting percentages in the premix were about 10% lower than cited in the premix recipe above.

200 ml of the resulting solution (Transparent 53, 6% liquid lecithin, 10.4 mg/ml mitragynine) was placed in a glass beaker on a magnetic stirrer and pH electrode was inserted to allow constant pH monitoring (initial pH was 4.1). To the constantly stirred solution, 10 N KOH was added dropwise, and 20 ml aliquots were drawn at the following pH: 4.1, 5.0, 6.0, 7.0, 8.9. The solution was monitored by visual observation to determine any change in turbidity and color. The first signs (slight increase of turbidity) were observed at pH above 7.0 and at pH 8.0 the solution was opaque indicating complete precipitation of Kratom extract. The aliquots were used for evaluation of organoleptic properties, measurements of Optical Density (300-800 nm), Particle Size (Dynamic Light Scattering, DLS), and entrapment efficiency (Dialysis-HPLC). All samples remained stable for at least 3 months, except the pH 8.9 which developed amorphous crystals growing on the tubes' walls and a very thin dark lipid-like film on top.

TABLE K1
Kratom precipitation pH in various formulations.

| formulation | Kratom extract precipitation pH |
|---|---|
| Zero sugar, no lecithin | 5.8 |
| Zero sugar, 6% liquid lecithin | 10.0 |
| Zero sugar, 4% de-oiled lecithin | 6.5 |
| Transparent 53, no lecithin | 6.2 |
| Transparent 53, 6% liquid lecithin | 8 |
| Transparent 53, 4% de-oiled lecithin | 7 |

Since transparent formulation 53 with de-oiled lecithin was developed and optimized with the intent to make the product visually appealing due to transparency, preparing this transparent formulation at pH above 6.5 makes no sense because it will lose its transparency and visual appeal. However, in the case of other formulations presented here, because at basic pH the organoleptic properties were improved, it does make sense to formulate at pH as high as 9.0, and potentially even higher if the taste improves further. It appears that neutral and basic pH may improve palatability through several mechanisms.

As already discussed, it helps masking sour/acidic off notes of Kratom extract. The exact molecular mechanism of such masking is not known but may be related to neutralization of the charge and reactivity of the tastants.

This example demonstrates that improvement of organoleptic properties of the extract, directly correlated with mitragynine complexation/encapsulation efficiency, which in turn, was increased by exposure of the encapsulation system to higher pH levels (Table K2).

One other aspect of the present teaching is the presence of oil in the system. Liquid lecithins are preparations of lecithins in oils where phospholipids make up about 50% of total mass. In the experiments presented here, liquid sunflower lecithin was used (Now Foods, 395 S. Glen Ellyn Rd., Bloomingdale, IL 60108) which is total phospholipids (mostly phosphatidyl choline with other phospholipids making less than 7% of total mass) dispersed in sunflower oil (triglycerides of mostly long chain oleic and linoleic acid). Mitragynine may incorporate more efficiently into formulations using liquid lecithin compared to de-oiled lecithin, which is likely the main contributor to improved organoleptic properties (Table K3). It is possible that the triglycerides become integrated in the liposomes and thus enhance integration of mitragynine and other tastants. However, the taste masking effect of the triglycerides themselves cannot be ruled out because mitragynine incorporation efficiency in the zero sugar de-oiled lecithin formulation is almost as high (78%) as in the zero sugar liquid lecithin formulation (88%) yet the organoleptic properties of the liquid lecithin formulation are significantly better, which is also in line with the improved organoleptic properties of the premix containing powdered oil in the dry powder Premix of Example M.

Finally, the most surprising observation from these experiments is further improvement of organoleptic properties upon precipitation of Kratom extract. On one hand the tastants precipitated into fine solid suspended particles, in which mitragynine is physically removed from the solution and may no longer interact with receptors. On the other hand, it is unexpected because the particles may become trapped in fine crevices of the tongue's surface, and dissolve with formation of concentrated solutions and leave a lasting unpleasant aftertaste that is very difficult to neutralize. Since in the present teachings the particles, clearly visible in some of the tested formulations, did not enhance the off-flavors but instead improved the taste, it appears that the generated Solid Lipid Nanoparticles are stable enough to be washed away with saliva, without dissolution on the tongue.

TABLE K2
Experimental Data.

| | | | Mitraginine | | | | Taste rating | |
|---|---|---|---|---|---|---|---|---|
| | | | incorporation | Effective | | Optical | within each | |
| Formulation | Lecithin | | efficiency, | diameter, | Poly- | density | set, 10-best, | |
| type | type | pH | % | nm | dispersity | at 600 nm | 1-worst | Physcial appearance |
| transparent | deoiled | 4.1 | 20 | 241 | 0.022 | 0.1012 | 1 | transparent |
| transparent | deoiled | 5.0 | not done | 244 | 0.097 | 0.1147 | 5 | transparent |
| transparent | deoiled | 6.0 | 22 | 266 | 0.153 | 0.1344 | 8 | transparent |
| transparent | deoiled | 7.0 | 50 | 339 | 0.285 | 0.2088 | 10 | translucent, aggregates |
| | | | | | | | | developed within days |
| transparent | deoiled | 8.0 | not done | 316 | 0.244 | 0.1967 | 8 | opaque, aggregates developed |
| | | | | | | | | within days |
| transparent | liquid | 4.1 | 46 | 317 | 0.08 | 0.3234 | 1 | translucent, opalescent |
| transparent | liquid | 5.0 | not done | 323 | 0.005 | 0.5677 | 3 | translucent, opalescent |
| transparent | liquid | 6.0 | not done | 318 | 0.005 | 0.4894 | 8 | translucent, opalescent |
| transparent | liquid | 7.0 | 84 | 330 | 0.005 | 0.9112 | 10 | translucent, became slightly |
| | | | | | | | | opaque within days |
| transparent | liquid | 8.9 | not done | not done | not done | not done | 10 | opaque |
| zero sugar | deoiled | 4.3 | 57 | 285 | 0.247 | 0.9527 | 1 | translucent, opalescent |
| zero sugar | deoiled | 5.0 | not done | 290 | 0.192 | 1.0318 | 4 | translucent, opalescent |
| zero sugar | deoiled | 6.0 | 73 | 350 | 0.257 | 1.3518 | 6 | translucent, opalescent |
| zero sugar | deoiled | 6.5 | not done | 459 | 0.268 | 2.3431 | 8 | opaque |
| zero sugar | deoiled | 7.0 | not done | 414 | 0.216 | 2.6869 | 10 | opaque |
| zero sugar | deoiled | 8.2 | not done | not done | not done | not done | 8 | opaque |
| zero sugar | liquid | 4.3 | 41 | 236 | 0.125 | 2.0727 | 1 | translucent, opalescent |
| zero sugar | liquid | 5.0 | not done | 166 | 0.132 | 2.3274 | 5 | translucent, opalescent |
| zero sugar | liquid | 6.0 | 69 | 247 | 0.216 | 2.0843 | 7 | translucent, opalescent |
| zero sugar | liquid | 7.0 | 88 | 225 | 0.005 | 2.784 | 10 | translucent, opalescent |
| zero sugar | liquid | 8.2 | not done | not done | not done | not done | 9 | translucent, opalescent |
| zero sugar | liquid | 9.0 | not done | not done | not done | not done | 8 | translucent, opalescent |
| zero sugar | liquid | 10.1 | not done | not done | not done | not done | 1 | opalescent, slightly opaque |

Samples were rated against each other by two trained panelists as described in other Examples. Liquid Kratom extract diluted in distilled water at 10 mg/ml was used as a base reference (10 out of 10 for aversiveness).

TABLE K3
Overall taste rating of formulations.

| | | 10 - highly aversive, |
|---|---|---|
| | | 0 - no off-flavors, |
| Formulation | pH | highly palatable |
| Transparent 53, 4% de-oiled lecithin | 7.0 | 3 |
| Transparent 53, 6% liquid lecithin | 7.0 | 2 |
| Zero sugar, 4% de-oiled lecithin | 7.0 | 4 |
| Zero sugar, 6% liquid lecithin | 7.0 | 1 |

Mitragynine encapsulation (incorporation/entrapment) efficiency was assessed by the dialysis membrane (DM) method as generally described in (Modi S, Anderson B D. Determination of drug release kinetics from nanoparticles: overcoming pitfalls of the dynamic dialysis method. Mol Pharm. 2013 Aug. 5; 10 (8): 3076-89). Dialysis Tubing 6.4 mm×10 mm, 12 kDa cutoff, product number IS13027, Innovating Science, Aldon Corporation was used. Briefly, 500 μl of sample was added to the bag, and then the bag was placed in 1 ml of water in a 1.5 ml vial and sealed. The vial was placed on a benchmark tube rocker and samples of the water outside the dialysis bag were collected at time zero and at 96 hours. Mitragynine concentration in the samples was measured by LC-MS. Control samples simulating 100% release (no encapsulation) were prepared by diluting 500 μl of the formulations directly into 1 ml of water (3× direct dilution).

Optical Density measurements were completed on all undiluted samples using a Varioskan™ LUX multimode microplate reader set for spectrum scan from 300-800 nm. OD₆₀₀ data was extracted and tabulated.

The Particle Size measurements were made using a Brookhaven Instruments 90 Plus Particle Size Analyzer set to: 25° C., angle: 90.00, run duration: 1.5 min. Refraction Index of Particles-Real: 1.590, Imaginary: 0.000, uniform Spheres and a dust cutoff of: 5000.00. All samples were diluted 10-fold in water to eliminate the effect of viscosity (confirmed by use of standard size particles).

### Example L

The following experiments demonstrate that phosphatidyl serine and phosphatidic acid may be used as alternatives to crude lecithins or combined with liquid or de-oiled lecithins to modify the formulations organoleptic, pharmacological, and physico-chemical characteristics. As mentioned above, lecithins carry their own off-flavors. Phosphatidic acid is one of the phospholipids that was demonstrated to be an efficient taste masker. Phosphatidyl serine is reported as taste masker for the first time.

In the first series of experiments, phosphatidic acid, phosphatidyl serine (Mediator 50P, and SerinAid 70P, Chemi Nutra, LLC, 11100 Metric Blvd., 200D, Austin, TX 78758) and phosphatidyl choline (Lipoid H 90, Lipoid GMBH, Ludwigshafen, Germany) were compared to liquid or de-oiled lecithins in two types of formulations-zero sugar, liquid lecithin (mostly aqueous) and transparent, de-oiled lecithin (less than 30% water).

### Premix for Zero Sugar Formulation Type

Masking agent OSF 7186B—0.5% w/v
Masking agent OSF 2070C—0.4% w/v
Masking agent MET 6985—2% w/v
Glycerol—10% w/v
Allulose—10% w/v
Sodium saccharin—0.05% w/v
Neohesperidin dihydrochalcone—0.025% w/v
Monk fruit extract, 25% mogroside V—0.15% w/v
Water—q.s. 900 ml

### Premix for Transparent Formulation Type

Glycerol—10% v/v
Propylene glycol—7.5% v/v
Ethanol—12.5% v/v
Masking agent OSF 7186B—0.5% w/v
Masking agent OSF 2070C—0.4% w/v
Masking agent MET 6985—2% w/v
Allulose—56% w/v
Sodium saccharin—0.025% w/v
Monk fruit extract, 25% mogroside V—0.15% w/v
Water—q.s. 1000 ml

Lecithins and phospholipids were added to 40 ml of the respective formulations and mixed vigorously for one hour, which ensured complete dissolution of the lecithins, whereas all phospholipids remained as either unstable emulsions or suspensions/dispersions.

Liquid Kratom stock solution: Dry powder extract with mitragynine content 70% (from Pure Kratom, LLC) was dispersed in a EGC solvent at a final concentration of mitragynine of 100 mg/ml. Heated to 70° C. to facilitate dissolution. Polysorbate 80 was added to a final concentration of 5.5%.

14 ml aliquots of samples were prepared by mixing the preparations of lecithins or phospholipids with liquid Kratom stock to yield 10 mg/ml concentration of mitragynine.

Samples were analyzed for organoleptic properties by two trained panelists as described in other Examples. The panelists were asked to rate the samples against each other with each formulation type and provide notes.

The formulations were evaluated by visual observation and kratom taste masking. Results of tests are shown in Table L1.

TABLE L1
Lecithins and individual phospholipids as taste maskers.

| Formulation | | | Kratom masking. |
|---|---|---|---|
| type | Phospholipid | Visual appearance | Overall rating and notes. |
| Zero sugar | Liquid lecithin, | Stable emulsion | Standard for this formulation |
| | 6% | | type. Second best. |
| zero sugar | PS 70, 4% | Suspension/dispersion | Best overall. Cleaner taste. |
| | | settling to the bottom | Reduced kratom taste, no |
| | | | bitterness, no irritation. |
| zero sugar | PC 90, 4% | Stable emulsion | Similar to lecithin but with more |
| | | | Kratom taste and bitterness. |
| zero sugar | PA 50, 4% | Suspension/dispersion | Similar to liquid lecithin. |
| | | settling to the bottom | |
| Transparent | De-oiled | Stable transparent | Standard for this formulation |
| | lecithin, 4% | emulsion | type. Second best. |
| Transparent | PS 70, 4% | Emulsion, lipids | Worst overall. More Kratom, |
| | | separating at the top | chemical taste, ethanol, lingering |
| | | | bitterness. |
| Transparent | PC 90, 4% | Stable emulsion | Similar to de-oiled lecithin. |
| Transparent | PA 50,4% | Emulsion, lipids | Best overall. |
| | | separating at the top | |

The results demonstrated that phospholipids performed differently in different formulation types. In a transparent formulation, phosphatidyl choline provided no advantage compared to de-oiled lecithin, which is expected, because de-oiled lecithin is mostly phosphatidyl choline. In the zero sugar type formulation it was inferior to liquid lecithin, potentially due to lack of the oil component.

Phosphatidic acid was superior to de-oiled lecithin and phosphatidyl serine in the transparent formulation. Whereas phosphatidyl serine was superior to phosphatidic acid and liquid lecithin in the zero sugar formulation.

Because individual phospholipids did not form stable emulsions on their own and the use of the individual phospholipids in Kratom shots will be too expensive, in the next series of experiments phosphatidic acid was combined with de-oiled lecithin for a transparent formulation and phosphatidyl serine with liquid lecithin for a zero sugar formulation. Several ratios and methods of mixing ratios were screened and arrived at the following two methods.

Liquid lecithin enriched with phosphatidyl serine was prepared by mixing 1500 g of liquid sunflower lecithin (Now Foods, 395 S. Glen Ellyn Rd., Bloomingdale, IL 60108) with 150 g of soybean 70% phosphatidyl serine Calcium salt powder (Lipoid PSP 70 a product of Lipoid GMBH, Ludwigshafen, Germany procured from American Lecithin Company, LLC). Mixing was performed for 8 hours using a planetary mixer (Kitchenaid). Homogeneity was confirmed by the absence of fine white specks of the phosphatidyl serine powder. Six grams of the resulting mix (comprised of ˜47% sunflower oil, ˜47% phosphatidyl choline, 6% phosphatidyl serine) was mixed with 80 ml of the zero sugar formulation overnight, and further combined with liquid Kratom stock, to yield a final concentration of 10 mg/ml mitragynine, 5.6% liquid lecithin, and 0.36% phosphatidyl serine. The resulting emulsion was stable, similar to that produced with liquid lecithin alone, and had reduced Kratom taste (sour notes), lingering bitterness and irritation compared to the standard formula containing 6% liquid lecithin alone. This indicated that liquid lecithin enriched with ˜ 6% of phosphatidyl serine can achieve the same masking effect at lower concentrations, resulting in fewer off-flavors carried by the liquid lecithin and reduced viscosity.

De-oiled lecithin enriched with phosphatidic acid was prepared by overnight mixing of 0.5 g of 50% phosphatidic acid (Mediator 50P, Chemi Nutra, LLC) with a standard transparent formulation containing 4% de-oiled lecithin. The mix was further combined with liquid Kratom stock, to yield a final concentration of 10 mg/ml mitragynine, ˜4% liquid lecithin, and 0.25% phosphatidic acid. The resulting emulsion was transparent and stable, similar to that containing de-oiled lecithin alone, and had reduced irritation and lingering bitterness compared to the de-oiled lecithin alone. This indicated that de-oiled lecithin enriched with 4% of phosphatidyl serine can achieve the same masking effect at lower concentrations resulting in fewer off-flavors, reduced viscosity, and improved transparency.

### Example M

The following experiments demonstrate that formulations of the present teaching effectively masked other off-flavors, not found in Kratom, hemp, mushrooms, or other botanicals presented in earlier examples. The experiment also demonstrates that multiple unpalatable active ingredients with different off-flavors can be effectively combined in one palatable product. The experiments further demonstrate preparation of liquid and powder premixes, consumable products, which can be used for preparation of palatable drinks comprising multiple active ingredients with different off-flavors. The unpalatable active ingredients were selected to simulate a sports nutrition drink.

The active ingredients and their sources are listed in Table M1.

TABLE M1
Active ingredients for sports nutrition drink,

| Nutritional Supplement | |
|---|---|
| Product name | Manufacturer or distributor |
| Potassium Chloride | Bulk Supplements (7511 Eastgate Rd, |
| | Henderson, NV 89011 |
| L-Leucine | Bulk Supplements |
| Guarana extract | Bulk Supplements |
| Creatine monohydrate | Bulk Supplements |
| Peavia ® ST80 | FINGRES BIOTECH INC. |
| Pea Protein 80% | Unit 1801, Coastal Center Economic |
| | Development Zone, Wuxi, Jiangsu |
| | 214125, China |
| AvailOm 50 High DHA Algae | Evonik Corporation, 2 Turner Place, |
| Algae oil-based, DHA as free | Piscataway, New Jersey 08854 |
| fatty acid min. 48 wt % | |
| AvailOm 50 High EPA | Evonik Corporation |
| Fish oil-based, EPA as free | |
| fatty acid min. 30 wt %, DHA | |
| as free fatty acid min. 15 wt % | |

The supplements were suspended in a premix or water for reference. The active ingredients and their dosages were selected to simulate a sports nutrition drink prepared in a small blender (200 ml volume).

Premix was prepared according to the following recipe.

Zero Sugar Formulation Type with Erythritol,
Liquid lecithin—6% w/v
Masking agent OSF 7186B—0.5% w/v
Masking agent OSF 2070C—0.4% w/v
Masking agent MET 6985—2% w/v
Erythritol—20% w/v
Sodium saccharin—0.05% w/v
Monk fruit extract, 25% mogroside V—0.15% w/v
Water—q.s. 1000 ml

Samples were analyzed for organoleptic properties by two trained panelists as described in other Examples. The panelists were asked to rate aversiveness/palatability and provide a brief description of off-flavors. Active ingredients, their concentrations, and the results are presented in Table M2.

TABLE M2
Premix vs water for taste masking of individual active ingredients.

| | dose, g | Taste in Water | Taste in Premix |
|---|---|---|---|
| Creatine | 5 | Strongly aversive, very | No bitterness. Aversive due |
| | | bitter, ammonia taste | to ammonia. |
| Magnesium Glycinate | 1.5 | Slightly aversive. Stale | Very palatable. No off- |
| | | moisture. | flavors. pH 10.3 caused |
| | | | lecithin color change. |
| KCl | 0.5 | Slightly aversive. Salty | Very palatable. No off- |
| | | and slightly bitter. | flavors. |
| L-Leucine | 5 | Strongly aversive, Strong, | Palatable. Slightly |
| | | characteristic BCAA | nutty/strawy, slightly chalky. |
| | | bitterness, irritation, | |
| | | metallic. | |
| Guarana extract | 0.5 | Aversive. Strongly bitter | Palatable. Very slight |
| | | and herbal. | bitterness. |
| Pea protein | 20 | Stale pea, vegetal, beany. | Palatable. Slightly beany. |
| Omega-3 and -6 | 2 | Aversive. Soapy, slightly | Palatable. No off-flavors. |
| | | bitter. Mildly algal/fishy. | |
| Omega-3 and -6 | 2 | Aversive. Soapy, slightly | Palatable. No off-flavors. |
| | | bitter. | |

The results demonstrate that the premix was efficacious in masking various off-flavors, except ammonia in Creatine. The current Creatine batch purchased from Bulk Supplements appeared to be of very low quality and unusually high pH, likely due to process related contaminants. The Chinese patent CN1240207A describes such a process of creatine manufacture (reaction between creatine sodium aqueous solution and a cyanamide aqueous solution) which uses ammonia at the last stage of production and has a high level of bitterant that requires additional purification step.

In the next experiment, Pea Protein—20 g, L-Leucine—5 g, KCl—0.5 g, Guarana extract—0.5 g, and 4 g of Omega-3 and -6, were blended into 200 ml of the premix containing either MET 6985 masker, as in typical Kratom formulations, or OSF 6754B PDR masker as in hemp formulation, Example D. The blends were taste tested side by side and both were found palatable. OSF 6754B PDR-containing blend was significantly more efficient at masking the BCAA irritation. Addition of Coffee flavor MET 17206 at 0.5% further improved palatability.

The composition of the liquid premix used for production of 200 ml of the blend with the listed active ingredients (Pea Protein—20 g, L-Leucine—5 g, KCl—0.5 g, Guarana extract—0.5 g, and 4 g of Omega-3 and -6), therefore, was as follows:

### Liquid Premix:

Liquid lecithin—12 g
Masking agent OSF 7186B—1 g
Masking agent OSF 2070C—0.8 g
Masking agent OSF 6754B PDR—1 g
Coffee flavor MET 17206-1 g
Erythritol—20 g
Sodium saccharin—0.1 g
Neohesperidin dihydrochalcone—0.05 g
Monk fruit extract, 25% mogroside V—0.3 g
Water—q.s 200 ml

In the next experiment a powder form premix, chemically and functionally similar to the liquid premix above, and enriched with phosphatidyl serine and phosphatidic acid to enhance taste masking capacity, was prepared according to the following recipe:

### Dry Powder Premix:

De-oiled lecithin—8 g
Powdered sunflower oil Nutri Sperse HOS 70ND—6 g
Phosphatidyl serine, 70%, PS 70-1 g
Phosphatidic acid 50%, PA 50-1 g
OSF 9918B PDR—1 g
OSF 9919B PDR—0.8 g
OSF 2683C PDR—1 g
OSF 6754B PDR—1 g
Coffee flavor MET 17206-1 g
Erythritol—20 g
Sodium saccharin—0.1 g
Neohesperidin dihydrochalcone—0.05 g
Monk fruit extract, 25% mogroside V—0.3 g

PA 50 and PS 70 are phosphatidic acid and phosphatidyl serine, respectively (Mediator 50P, and SerinAid 70P, Chemi Nutra, LLC). Nutri Sperse HOS 70ND is a powder form of high oleic oil from sunflower (Abitec, 501 West 1 Ave, Columbus, OH 43215). OSF 9918B PDR is a powder equivalent of OSF 7186B used in liquid premixes. OSF 2683C PDR and OSF 9919B PDR together make an equivalent of OSF 2070C used in liquid premixes.

The dry premix was poured into a kitchen blender, combined with 200 ml of water and the sports nutrition supplements as in the previous experiment of this example (Pea Protein—20 g, L-Leucine—5 g, KCl—0.5 g, Guarana extract—0.5 g, and 4 g of Omega-3 and -6) and blended for about 20 seconds. The resulting blend of dry premix, water, and the nutritional supplements was a moderately viscous, opaque, stable emulsion, similar in its appearance to the blend where the liquid premix was used. The blend was taste tested immediately after the preparation and after 8-hour incubation at +4° C. or +25° C., and compared to the blend prepared with the liquid premix. The powder premix derived blend, immediately after the blending, displayed significantly more off-flavors and irritation compared to the liquid premix derived blend. The off-flavors and were attributed to vegetal and beany notes of pea protein, a characteristic BCAA taste, and epiglottis irritation by BCAA. The powder premix derived blends also lacked sweetness, especially the lingering sweetness of Neohesperidin Dihydrochalcone. This sweetener is known to have low water solubility and, in the liquid formulations of the present teaching, it was used as a 10% w/v stock solution in propylene glycol. Preparation of such PG stock, which is stable for weeks, or a hot water stock, which is stable only for a few hours before it precipitates, is a standard industry practice. The powder premix derived blends incubated for 8 hours, either at +4° C. or 25° C., were almost indistinguishable from the liquid premix derived blend. This confirms that proper hydration of individual phospholipids and de-oiled lecithin, which may take several hours, allows for full taste masking potential. The 8-hour incubated blends also gained in sweetness and lingering sweetness suggesting that dry Neohesperidin Dihydrochalcone was fully dissolved during the incubation.

A sample not containing powdered sunflower oil (Nutri Sperse HOS 70ND) was also prepared and analyzed as above. This sample showed inferior organoleptic properties compared to the one with powdered oil, confirming observations in Example K, that presence of non-phospholipid oils or fats contributes to taste masking properties of the formulations and premixes of present teaching. All dry premix derived samples and their palatability ratings are summarized in Table M3.

TABLE M3
Parameters affecting taste masking capacity of dry powder premix.

| | | | | | Palatability |
|---|---|---|---|---|---|
| Incubation | Incubation | | Nutri Sperse | Coffee | 1-worst, 5-best |
| time | temperature | | HOS 70ND | flavor | (highly palatable) |
| 0 | N/A | | no | no | 1 |
| 8 hours | +4° | C. | no | no | 2 |
| 8 hours | +25° | C. | no | no | 2 |
| 8 hours | +4° | C. | no | yes | 3 |
| 0 | N/A | | yes | no | 2 |
| 8 hours | +4° | C. | yes | no | 4 |
| 8 hours | +25° | C. | yes | no | 4 |
| 8 hours | +4° | C. | yes | yes | 5 |

Other functionalities can be added to the liquid or dry premixes. In one aspect of the present teaching, the premix also includes a buffering system to neutralize basic or acidic compounds. In another aspect of the present teaching, the premix includes a premixed flavor. In Example M a Coffee flavor was used, other matching flavors may be selected as shown in other Examples B and D. In another aspect of the present teaching, phosphatidic acid, phosphatidyl serine, phosphatidyl inositol, other phospholipids, or their combinations are added to further enhance taste masking properties of the premix as may be required in extreme applications as taught in the Example N.

The liquid and dry premixes can also incorporate food colors, preservatives, processing aids, and other excipients and functional ingredients that enhance user experience, and product stability, shelf life, and manufacturability.

The unpalatable active ingredients and dosages of Example N were selected to simulate a sports nutrition drink prepared in a small blender (200 ml volume). However, it should be understood that a drink with any other functional nutrition, supplements, or pharmaceuticals, in different combinations and dosages, may be made palatable using the approach described. In one aspect of the present teaching, the products requiring taste masking are foods, nutritional supplements, or pharmaceuticals in the form of powders, solids (including pills, capsules, tablets), semi-solids, or liquids. The end user may choose to blend pills, capsules, tablets, powders, liquids, etc., into one palatable drink. Many health-conscious people today take 20-40 supplements daily, typically as capsules and loose, frequently unpalatable, powders, which is very inconvenient. The nutritional supplement industry acknowledges that there is an increasing trend to take supplements in forms alternative to solid dosage forms (pills, capsules, and tablets). This approach is also useful for dosing infants who cannot swallow capsules and adults who have swallowing problems (dysphagia).

### Example N

This experiment intends to describe dosing of a patient with a pharmaceutical delivered in a sublingual dosage form.

Ajmalicine (Raubasine) is available from Sigma-Aldrich and dissolved in EGP solvent at a final concentration of 180 mg/ml. 0.5 ml of the solution can be mixed with 4.5 ml of the premix (any of the Premixes listed in Example K, optionally with sugar free liquid lecithin Premix).

The resulting formulation may be adjusted to pH between 5 and 10 and administered to a hypertension patient with constant blood pressure monitoring. The patient is instructed to keep the dosage under the tongue, without swallowing it for as long as possible.

Other liquid, solid, or semisolid dosage forms can be formulated using the same general taste masking approach. Examples of such products include gels, pastes, lozenges, gummies, lollipops, troches, pastilles, chewing gums, oral films, and the like.

### Example O

Non-stable suspension with added botanicals.

30 g of Aloe vera juice, RealAloe (Real Aloe Solutions, 7470 S. Dean Martin Dr., Suite 102, Las Vegas, NV 89139) was mixed with 1.5 g of liquid sunflower lecithin (Now Foods, 395 S. Glen Ellyn Rd., Bloomingdale, IL 60108) to produce a homogeneous emulsion. 2.2 g of sucrose was added, and the emulsion was flavored with 150 mg of Masking agent OSF 7187, 120 mg of Masking agent OSF 2070C (OSF Flavors, 40 Baker Hollow Rd, Windsor, CT 06095), 600 mg of Masking agent MET 6985, and 210 mg of Passion Fruit flavor MET09667 (Foodarom, 5400 Rue Armand-Frappier, Saint-Hubert, QC J3Z 1G5, Canada).

The resulting solution was mixed with 10 ml of liquid Kratom stock prepared by dissolving 70% mitragynine powder (a sample from PDX Aromatix) in 50% w/v Sucrose, 0.5% ascorbic acid, in water, at a final concentration of 30 mg/ml.

The resulting formulation, containing 7.5 mg/ml mitragynine, was not stable and separated into several distinct layers in a matter of hours. The resulting formulation was homogenized by brief shaking prior to taste testing and will start separating within an hour. The lecithin-containing formulation demonstrated dramatically improved palatability compared to a control not containing any lecithin. Similar results were obtained when de-oiled soybean lecithin (Quality Supplements and Vitamins, Inc. Ft. Lauderdale, FL, 33309) was used.

The emulsion was partially stabilized by Polysorbate 80 at 1% w/v.

This experiment demonstrates that phase stability of lecithin emulsions may not be required to employ taste masking properties of phospholipids.

### Example P

A formula for manufacture of eatable, solid dosage form-protein bar containing 150 mg of mitragynine and 20 g of whey and milk proteins in a 57 g bar.

A protein bar base was prepared using the ingredients listed in Table P1. A liquid mix (maskers, flavors, and HIS in water) was mixed with the Powder mix until homogenized, and the resulting dough was mixed with the fats.

Liquid Kratom stock solution. Dry powder extract with mitragynine content 70% (from Pure Kratom, LLC) was dispersed in EGP solvent at final concentration of mitragynine 100 mg/ml and heated to 70° C. to facilitate dissolution. Polysorbate 80 was added to a final concentration of 5.5% w/v.

Liquid Kratom stock was used either as is (pH 3.4, 1.5 ml, 150 mg mitragynine) or neutralized with 10 N KOH (pH 7.0, ˜1.6 ml, 150 mg mitragynine). Liquid Kratom stock was added to either the liquids or to the fats to determine if mixing matrix affected organoleptic properties.

One sample was prepared by mixing 1.5 ml of liquid Kratom into fats first and then mixing a predetermined amount (0.115 ml) of 10M KOH needed to achieve pH 7.0. Such an approach was expected to improve complexation of mitragynine and other tastants of Kratom with phospholipids.

TABLE P1
Protein bar recipe.

| | | |
|---|---|---|
| Ingredient | Amount, g | Manufacturer or Distributor |

Powder mix

| | | |
|---|---|---|
| BarPro, milk protein isolate | 13.4 | Glanbia Nutritionals, 2840 Loker Ave E, |
| powder | | Carlsbad, CA 92010 |
| BarFlex, whey protein isolate | 7.4 | Glanbia Nutritionals |
| powder | | |
| AdvantaThrive 90, Isomalto- | 12 | Top Health Ingredients, 1580-5555 Calgary Tr. |
| oligosaccharides powder | | NW, Edmonton, AB, T6H 5P9, CANADA |
| PolySweet, erythritol fine | 5 | Anderson Advanced Ingredients, 2030 Main St |
| powder | | #430, Irvine, CA 92614 |

Liquid mix

| | | |
|---|---|---|
| Water | 3.8 | |
| Neohesperidin | 0.02 | Bordas, Calle Acueducto No 4-6, Polígono |
| Dihydrochalchone | | Industrial La Isla 41703, Dos Hermanas, Sevilla, |
| | | Spain |
| Sodium Saccharin | 0.04 | NiuSource, Inc., 14266 Euclid Ave, Chino, CA |
| | | 91710 |
| Monk fruit extract, 25% | 0.12 | Icon Foods, 19250 NE Portal Way, Portland, OR |
| mogroside V | | 97230 |
| Guanabana flavor MET 20783 | 0.3 | Foodarom, 5400 Rue Armand-Frappier, Saint- |
| | | Hubert, QC J3Z 1G5, Canada |
| Masker MET 6985 | 1.2 | Foodarom |
| Masker OSF 7186B | 0.3 | OSF Flavors, 40 Baker Hollow Rd, Windsor, CT |
| | | 06095 |
| Masker OSF 2070C | 0.24 | OSF Flavors |

Fat mix

| | | |
|---|---|---|
| raw cashew butter | 7 | Blue Mountain Organics Distribution, LLC, |
| | | P.O. Box 898 Floyd, VA 24091 USA |
| liquid lecithin | 2.6 | Now Foods, 395 S. Glen Ellyn Rd., |
| | | Bloomingdale, IL 60108 |
| Bio PS 20% Liquid, 20% | 2 | ECA Healthcare, 1260 E Locust St, Ontario, CA |
| phosphatidyl serine in MCT oil | | 91761 |

A total of five preparations were evaluated.
1) pH 7 Kratom, mixed with fats
2) pH 7 Kratom, mixed with liquids
3) pH 3.4 Kratom, mixed with fats
4) pH 3.4 Kratom, mixed with liquids
5) pH 3.4 Kratom, mixed with fats, then neutralized

Note that the final pH of the protein bar samples 3 and 4 was 5.4, likely due to buffering capacity of milk and whey proteins. pH measurement was done on 1 g of final preparation dispersed in 4 ml of distilled water.

Two panelists chewed 7 g portions for 15 seconds before expectoration. Panelists were asked to rate the samples against each other and provide comments on palatability and off flavors. All samples were described as highly palatable and had subtle but noticeable differences. Samples 3 and 4 possessed minor irritation. Sample 5 was reported as the best overall, suggesting that neutralization in the presence of phospholipids and fats, similar to Example K, may improve formation of complexes between phospholipids/fats and tastants in Kratom extract.

Formulation of edible solid dosage forms typically presents a higher palatability masking challenge than liquid forms. This experiment clearly demonstrates that the taste masking system (sweetener system, masking agents, and phospholipids) of the present teaching is directly applicable to solid dosage forms. Other edible products in solid or semisolid form can be formulated using the same general approach. Examples of such products include lozenges, gummies, lollipops, troches, pastilles, chewing gums, oral films, and the like.

### Example Q

This example demonstrates that it may be possible to replace the sweetener system, partially or entirely, with a taste modulator, such as Synsepalum dulcificum fruit, its preparations, or miraculin protein. By using this approach, a zero sweetener Kratom product may be created.

The experiment was designed to evaluate the effect of S. dulcificum on taste of Kratom in the context of lecithin-containing formulation.

Liquid Kratom stock solution. Dry powder extract with mitragynine content 70% (from Pure Kratom, LLC) was dispersed in EGP solvent at final concentration of mitragynine 100 mg/ml and heated to 70° C. to facilitate dissolution. Polysorbate 80 was added to a final concentration of 5.5% w/v.

The same general zero sugar premix used in other experiments was used with reduced amount of Allulose and Glycerol to further minimize their effect on sweetness. Four variations of zero sugar premix were prepared as shown in Table Q1.

### Zero Sugar Premix

Liquid lecithin—6% w/v
Masking agent OSF 7186B—0.5% w/v
Masking agent OSF 2070C—0.4% w/v
Masking agent MET 6985—2% w/v
Glycerol—5% w/v
Allulose—5% w/v
Sodium saccharin—0.05% w/v
Monk fruit extract, 25% mogroside V—0.15% w/v

TABLE Q1
Zero sugar premix variations.

| | | Lecithin and | Lecithin and | Complete |
|---|---|---|---|---|
| | Lecithin only | Maskers | Sweeteners | premix |
| lecithin | ✓ | ✓ | ✓ | ✓ |
| OSF 7186B | | ✓ | | ✓ |
| OSF 2070C | | ✓ | | ✓ |
| MET 6985 | | ✓ | | ✓ |
| saccharin | | | ✓ | ✓ |
| Neohesperidin | | | ✓ | ✓ |
| Monk fruit | | | ✓ | ✓ |

The four premixes were combined with liquid Kratom stock to yield 10 mg/ml mitragynine. A sample of Kratom in water at the same concentration was used as a reference. Two panelists were asked to taste test the Kratom in water reference and then to taste test the samples and provide the scores for bitterness, sour/acid notes (0—not present, 10—as in Kratom water reference) rate overall palatability and provide notes where applicable.

After the four samples were tested, the panelists were asked to chew one lyophilized miracle berry Richberry® (Miracu Lean, Barangay Hiwacloy, 4422, Camarines Sur, Philippines) for 30 seconds, rinse the mouth with water and repeat taste test on the same samples as usual. The results of taste test are shown in Table Q2.

TABLE Q2
Off-flavors and palatability of Kratom in Premix variations
before and after ingestion of miracle berry.

| | | | |
|---|---|---|---|
| | Lecithin and | Lecithin and | Complete |
| Lecithin only | Maskers | Sweeteners | premix |

Before miracle berry

| | | | | |
|---|---|---|---|---|
| Bitterness/ | 6 | 2 | 3 | 1 |
| aftertaste bitterness | 6 | 2 | 4 | 0 |
| Sour/acidic/Kratom | 6 | 5 | 5 | 2 |
| Palatability | Unpalatable | Unpalatable | Unpalatable | Highly palatable |

After miracle berry

| | | | | |
|---|---|---|---|---|
| Bitterness/ | 2 | 1 | 2 | 0 |
| aftertaste bitterness | 2 | 0 | 2 | 0 |
| Sour/acidic/Kratom | 2 | 1 | 2 | 1 |
| Palatability | Palatable | Highly palatable | Palatable | Highly palatable |

The panelists noticed that compared to standard formulations, all samples, and especially the ones lacking all maskers, produced higher irritation and tongue burning, likely due to lower concentrations of LIS (glycerol and allulose in this case). Miracle berry was very efficient at converting the sour/acidic Kratom notes into sweetness and noticeably masked the bitterness.

Having thus described the present teaching, it is claimed:

1-20. (canceled)

21. A liquid, oral formulation comprising:
Kratom;
liquid lecithin or a combination of liquid and de-oiled lecithin;
at least one masking agent;
at least one flavor; and
at least two sweeteners;
wherein the pH of the formulation is greater than 5.0.

22. The formulation of claim 21, further comprising an emulsifier.

23. The formulation of claim 22, wherein said emulsifier is a polysorbate.

24. The formulation of claim 21, wherein the formulation further comprises phosphoric acid.

25. The formulation of claim 21, wherein the masking agent is selected from the group consisting of a flavanone glycoside, eriodictyol, homoeriodictyol, and combinations thereof.

26. The formulation of claim 25, wherein the flavanone glycoside is neohesperidin dihydrochalchone.

27. The formulation of claim 21, wherein the formulation is devoid of sucralose and stevia.

28. The formulation of claim 21, wherein the at least two sweeteners comprise a low intensity sweetener.

29. The formulation of claim 28, wherein the low intensity sweetener is selected from the group consisting of sucrose, fructose, tagatose, erythritol, allulose, sorbitol, xylitol, glycerol, sorbose, and allose.

30. The formulation of claim 21, wherein the at least two sweeteners comprise a high intensity sweetener.

31. The formulation of claim 30, wherein the high intensity sweetener is selected from the group consisting of saccharin, aspartame, acesulfame K, neotame, and monk fruit.

32. The formulation of claim 21, wherein the at least two sweeteners comprise a high intensity sweetener and a low intensity sweetener.

33. The formulation of claim 21, wherein the formulation further comprises phyllodulcin.

34. The formulation of claim 21,
wherein the masking agent is selected from the group consisting of a flavanone glycoside, eriodictyol, homoeriodictyol, and combinations thereof;
wherein the at least two sweeteners comprise 1) a high intensity sweetener selected from the group consisting of saccharin, aspartame, acesulfame K, neotame, and monk fruit and 2) a low intensity sweetener selected from the group consisting of sucrose, fructose, tagatose, erythritol, allulose, sorbitol, xylitol, glycerol, sorbose, and allose;
wherein the formulation further comprises phosphoric acid; and
wherein the formulation further comprises a polysorbate.

35. The formulation of claim 34, wherein the masking agent is a flavanone glycoside.

36. The formulation of claim 35, and wherein the flavanone glycoside is neohesperidin dihydrochalchone.

37. The formulation of claim 34, wherein the formulation is devoid of sucralose and stevia.

38. The formulation of claim 34, wherein the formulation further comprises phyllodulcin.

39. The formulation of claim 34, wherein the flavor is caramel.
